(19) United States
(12) Patent Application Publication
(10) Pub. No.: US 2026/0042873 A1
(43) Pub. Date: Feb. 12, 2026

(54) POLYMER BRUSHES WITH CHAIN-ENDS FUNCTIONALIZED WITH METAL COORDINATING TWO HETERO ELEMENTS FOR SELECTIVE SURFACE MODIFICATION

(71) Applicant: Merck Patent GMBH, Darmstadt (DE)

(72) Inventors: Md S. Rahman, Flemington, NJ (US); Sachin Bobade, Branchburg, NJ (US); EunJeong Jeong, Gyeonggi-do (KR); Edward W. Ng, Belle Mead, NJ (US); Durairaj Baskaran, Bridgewater, NJ (US)

(21) Appl. No.: 19/099,935

(22) PCT Filed: Aug. 15, 2023
(86) PCT No.: PCT/EP2023/072428

§ 371 Date: Jan. 30, 2025

Related U.S. Application Data
(60) Provisional application No. 63/371,670, filed on Aug. 17, 2022.

(51) Int. Cl. C08F 8/40; C08F 8/34; C09D 125/06; C09D 133/12

(52) U.S. Cl. CPC C08F 8/40; C08F 8/34; C09D 125/06; C09D 133/12; C08F 2810/40

(57) ABSTRACT

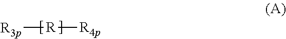

A polymer of structure (A) with two end group R₃ₚ, and R₄ₚ, and a polymer chain (R) which has either repeat units of structures (Ip) or (IIp). R₃ₚ, is either a C-1 to C-8 alkyl, a moiety of structure (IIIp), or a moiety of structure (IIIpI). R₃ₚ is a C-1 to C-8 alkyl if the repeat unit is (IIp). R₃ₚ is structure (IIIp) or structure (IIIp1) if the repeat unit has (Ip). R4p is either a terminating moiety such as H, a moiety of structure (IVp), a moiety of structure (IVp1), or a moiety of structure (IVp2). R₃ₚ and R₄ₚ cannot respectively, both simultaneously, be moieties of structure (IIIp) and (IVp), simultaneously be moieties of structure (IIIp) and (IVp1), or simultaneously be moieties of structure (IIIp) and (IVp2). The polymer of structure (A) must contain, one grafting end group moiety selected from structures (IIIp), (IVp), (IVp1) or (IVp2). Composition of this with a solvent are used in DSA processing.

### FIELD

The disclosed subject matter pertains to novel graftable polymers with narrow polydispersity and one reactive end group selective to metal substrates which contains two heteroatoms and compositions thereof in an organic spin casting solvent and the process of forming polar or non-polar brushes on a substrate using these compositions and using these brushes for directed self-assembly. Also disclosed are novel compounds useful in making these polymers. The invention also relates to the process of forming polar or non-polar brushes on a substrate using these compositions and using these brushes for directed self-assembly (DSA).

### RELATED ART

In conventional lithography approaches, ultraviolet (UV) radiation may be used to expose through a mask onto a photoresist layer coated on a substrate or layered substrate. Positive or negative photoresists are useful, and these can also contain a refractory element such as silicon to enable dry development with conventional integrated circuit (IC) plasma processing. In a positive photoresist, UV radiation transmitted through a mask causes a photochemical reaction in the photoresist such that the exposed regions are removed with a developer solution or by conventional IC plasma processing. Conversely, in negative photoresists, UV radiation transmitted through a mask causes the regions exposed to radiation to become less removable with a developer solution or by conventional IC plasma processing. An integrated circuit feature, such as a gate, via or interconnect, is then etched into the substrate or layered substrate, and the remaining photoresist is removed. When using conventional lithographic exposure processes, the dimensions of features of the integrated circuit feature are limited. Further reduction in pattern dimensions is difficult to achieve with radiation exposure due to limitations related to aberrations, focus, proximity effects, minimum achievable exposure wavelengths and maximum achievable numerical apertures. Directed self-assembly is a promising approach which has been of interest in overcoming some of the drawback of conventional lithography as outlined above.

Specifically, directed self-assembly of block copolymers is a method useful for generating very small, patterned features for the manufacture of microelectronic devices in which the critical dimensions (CD) of features usually on the order of nano scale ranging in feature size from 10 nm to 50 nm can be achieved. Achieving feature sizes below 10 nm using conventional approaches for directed self-assembly of block copolymers is challenging. Directed self-assembly methods such as those based on graphoepitaxy and chemical epitaxy of block copolymers are desirable for extending the resolution capabilities of lithographic technology.

These techniques can be employed to either enhance conventional lithographic techniques by enabling the generation of pattern with higher resolution and/or improving CD control for EUV, e-beam, deep UV or immersion lithography. The directed self-assembly block copolymer comprises a block of etch resistant polymeric unit and a block of highly etchable polymeric unit, which when coated, aligned and etched on a substrate give regions of high-resolution patterns.

Known examples of block copolymers suitable for directed self-assembly are ones capable of microphase separation and comprising a block rich in carbon (such as styrene or containing some other element like Si, Ge, and Ti) which is resistant to plasma etch, and a block which is highly plasma etchable or removable, which can provide a high-resolution pattern definition. Examples of highly etchable blocks can comprise monomers which are rich in oxygen, and which do not contain refractory elements and are capable of forming blocks which are highly etchable, such as methyl methacrylate. The plasma etching gases used in the etching process of defining the self-assembly pattern typically are those used in processes to make integrated circuits (IC). In this manner very fine patterns can be created on typical IC substrates compared to conventional lithographic techniques, thus achieving pattern multiplication.

In the graphoepitaxy directed self-assembly method, the block copolymers self-organize on a substrate that is pre-patterned with conventional lithography (Ultraviolet, Deep UV, and e-beam, Extreme UV (EUV) exposure source) to form topographical features such as a line/space (L/S) or contact hole (CH) pattern. In an example of L/S directed self-assembly array, the block copolymer can form self-aligned lamellar regions with a sub-lithographic pitch in the trenches between sidewalls of pre-pattern, thus enhancing pattern resolution by subdividing the space in the trench between the topographical lines into finer patterns. Similarly, features such as contact holes can be made denser by using graphoepitaxy in which a suitable block copolymer arranges itself by directed self-assembly within an array of pre-patterned holes or pre-patterned posts defined by conventional lithography, thus forming a denser array of regions of etchable and etch resistant domains which when etched give rise to a denser array of contact holes. In addition, block copolymers can form a single and smaller etchable domain at the center of prepattern hole with proper dimension and provide potential shrink and rectification of the hole in prepattern. Consequently, graphoepitaxy has the potential to offer both pattern rectification and pattern multiplication.

In chemical epitaxy (chemoepitaxy) DSA methods, the self-assembly of the block copolymer occurs on a surface that has regions of differing chemical affinity but no or very slight topography to guide the self-assembly process. For example, the chemical prepattern could be fabricated using lithography (UV, Deep UV, e-beam, EUV) and nanofabrication process to create surfaces of different chemical affinity in a line and space (L/S) pattern. These areas may present little to no topographical difference but do present a surface chemical pattern to direct self-assembly of block copolymer domains. This technique allows precise placement of these block copolymer domains of higher spatial frequency than the spatial frequency of the prepattern. The aligned block copolymer domains can be subsequently pattern transferred into an underlying substrate after plasma or wet etch processing. In addition, Chemical epitaxy has the advantage that the block copolymer self-assembly can rectify variations in the surface chemistry, dimensions, and roughness of the underlying chemical pattern to yield improved line-edge roughness and CD control in the final self-assembled block copolymer domain pattern. Other types of patterns such as contact holes (CH) arrays could also be generated or rectified using chemoepitaxy.

The ability of a BCP to phase separate depends on the Flory Huggins interaction parameter (W). PS-b-PMMA (poly(styrene-block-methyl methacrylate) is the most promising candidate for directed self-assembly (DSA) applications. However, the minimum half-pitch of PS-b-PMMA is limited to about 10 nm because of lower interaction parameter (A) between PS and PMMA. To enable further feature miniaturization, a block copolymer with a larger interaction parameter between two blocks (higher chi) is highly desirable.

For lithography applications, orientation of the block copolymer domains perpendicular to the substrate is desirable. For a conventional block copolymer such as PS-b-PMMA in which both blocks have similar surface energies at the BCP-air interface, this can be achieved by coating and thermally annealing the block copolymer on a layer of non-preferential or neutral material that is grafted or cross-linked at the polymer-substrate interface. Due to larger difference in the interaction parameter between the domains of higher-Chi block copolymers, it is important to control both BCP-air and BCP-substrate interactions. Many orientation control strategies for generating perpendicularly oriented BCP domains have been implemented with higher-Chi BCPs. For example, solvent vapor annealing has been used for orientation control of polystyrene-b-polyethylene oxide (PS-b-PEO), polystyrene-b-polydimethylsiloxane (PS-b-PDMS), polystyrene-b-poly(2-vinyl pyridine) (PS-b-P2VP), polylactide-b-poly(trimethylsilylstyrene) PLA-b-PTMSS and PDMS-b-PHOST. Introducing a solvent vapor chamber and kinetics of solvent vapor annealing may complicate DSA processing. Alternatively, the combination of neutral underlayers and topcoat materials has been applied to PS-b-P2VP, PS-b-PTMSS and PLA-b-PTMSS to achieve perpendicular orientation of the polymer domains. However, the additional topcoat materials may increase the process cost and complexity. Thus, there exists a need to have a topcoat free higher-Chi BCP system using simple thermal annealing on a range of preferential and non-preferential substrates.

Grafted polymer which forms covalently bound film on the surface of a substrate can be prepared by plasma deposition, electrochemical deposition or self-assembly. The strength of covalent bond predicates the adherence of film; however, these films are generally much more adherent than films which only interact through secondary forces with the surface of the substrate such as those prepared by spin casting. Consequently, because of this higher adherence formation of a grafted polymer film on a substrate material is useful for a variety of applications. Among these are the following examples: One example are biomaterials where substrates are made bio compatible by grafting a polymer at the surface of a material, such as medical prostheses, without compromising bulk mechanical properties. Another example are polymers on substrate surfaces also has been employed to impart anti-bio fouling of these surfaces or to improve their corrosion resistance. Another example are coating solutions, where the grafting of a polymer on a substrate surface can change the surface properties of these substrates to affect better coating; also, in suspension of metal or metal oxide nanoparticles the coating ability and stability of these suspensions may be improved by the grafting of polymers at the surface of these nanoparticles. Other examples self-assembly and directed self-assembly, where the grating of polymer brushes on the surface of Silicon or Silicon oxide substrates can be employed for the formation of neutral layer on these surfaces which allow block copolymer to orient their domains perpendicular to the substrate surface during self-assembly or directed self-assembly.

Directed self-assembly of block copolymers is a method useful for generating smaller and smaller patterned features for the manufacture of microelectronic devices in which the critical dimensions (CD) of features on the order of nanoscale can be achieved. Directed self-assembly methods are desirable for extending the resolution capabilities of microlithographic technology. In a conventional lithography approach, ultraviolet (UV) radiation may be used to expose through a mask onto a photoresist layer coated on a substrate or layered substrate. Positive or negative photoresists are useful, and these can also contain a refractory element such as silicon to enable dry development with conventional integrated circuit (IC) plasma processing. In a positive photoresist, UV radiation transmitted through a mask causes a photochemical reaction in the photoresist such that the exposed regions are removed with a developer solution or by conventional IC plasma processing. Conversely, in negative photoresists, UV radiation transmitted through a mask causes the regions exposed to radiation to become less removable with a developer solution or by conventional IC plasma processing. An integrated circuit feature, such as a gate, via or interconnect, is then etched into the substrate or layered substrate, and the remaining photoresist is removed. When using conventional lithographic exposure processes, the dimensions of features of the integrated circuit feature are limited. Further reduction in pattern dimensions is difficult to achieve with radiation exposure due to limitations related to aberrations, focus, proximity effects, minimum achievable exposure wavelengths and maximum achievable numerical apertures. The need for large-scale integration has led to a continued shrinking of the circuit dimensions and features in the devices. In the past, the final resolution of the features has been dependent upon the wavelength of light used to expose the photoresist, which has its own limitations. Direct assembly techniques, such as graphoepitaxy and chemoepitaxy using block copolymer imaging, are highly desirable techniques used to enhance resolution while reducing CD variation. These techniques can be employed to either enhance conventional UV lithographic techniques or to enable even higher resolution and CD control in approaches employing EUV, e-beam, deep UV or immersion lithography. The directed self-assembly block copolymer comprises a block of etch resistant copolymeric unit and a block of highly etchable copolymeric unit, which when coated, aligned and etched on a substrate give regions of very high-density patterns.

Neutral layers are layers on a substrate or the surface of a treated substrate which have no affinity for either of the block segment of a block copolymer employed in directed self-assembly. In the graphoepitaxy method of directed self-assembly of block copolymer, neutral layers are useful as they allow the proper placement or orientation of block polymer segments for directed self-assembly which leads to proper placement of etch resistant block polymer segments and highly etchable block polymer segments relative to the substrate. For instance, in surfaces containing line and space features which have been defined by conventional radiation lithography, a neutral layer allows block segments to be oriented so that the block segments are oriented perpendicular to the surface of the substrates, an orientation which is ideal for both pattern rectification and pattern multiplication depending on the length of the block segments in the block copolymer as related to the length between the lines defined by conventional lithography. If a substrate interacts too strongly with one of the block segments it would cause it to lie flat on that surface to maximize the surface of contact between the segment and the substrate; such a surface would perturb the desirable perpendicular alignment which can be used to either achieve pattern rectification or pattern multiplication based on features created through conventional lithography. Modification of selected small areas or pinning of substrate to make them strongly interactive with one block of the block copolymer and leaving the remainder of the surface coated with the neutral layer can be useful for forcing the alignment of the domains of the block copolymer in a desired direction, and this is the basis for the pinned chemoepitaxy or graphoepitaxy employed for pattern multiplication.

There is a need for a novel materials which can form a grafted polymer layer on semiconductor (e.g. Si, GaAs, and the like), metal (Cu, W, Mo, Al, Zr, Ti, Hf, Au and the like) and metal oxide (Copper oxide, Aluminum oxide, Hafnium oxide, Zirconium oxide, Titanium oxide and the like) substrates through a simple spin coating, followed by a post coat bake to affect chemical bonding without the presence of activating components to promote the grafting reaction on the substrate such as acidic compounds, thermal acid generators, photoacid generator, thermal radical generators, photochemical radical generators, basic additives, thermal base generators or photobase generators. The presence of such thermally or photochemically reactive additives compounds is undesirable because the small size and reactivity of these compounds, they may lead them to diffuse out of the grafted film into other layers causing undesirable reaction such as corrosion. Another need is for a grafting material in which graftable polymer does not contain overly reactive grafting sites which may deleteriously affect shelf life of solutions of a grafting solution in an organic solvent such as a spin casting solvent. There is also a need for novel grafting material than can be made to have selective grafting towards specific types of substrates by altering grafting bake. In this manner one can alter the surface properties of these materials, such as coat-ability, and corrosion resistance by a simple spin coating process without having to use plasma deposition or electrochemical grating, and also in the case of a novel selective grafting process using the novel materials of this invention, in one step coat only one type of material on a substrate which contains a topographical or chemical pattern in which different materials are present on one substrate. There is also a need for novel neutral layer compositions which when formed into a layer remain neutral to the self-assembly block copolymer and yet are not damaged by processing steps of directed self-assembly techniques and can further enhance the lithographic performance of the directed self-assembly materials and processes, especially reducing the number of processing steps and providing better pattern resolution with good lithographic performance. There is also a need for coat-able pinning materials for small areas of metal or metal oxide substrates otherwise coated with a neutral layer, for instance in the chemoepitaxy approach, in order to force the domains oriented perpendicularly with the neutral layer substrate to force the alignment of the domains in a desired direction. There is also a need for a polar and non-polar brush compositions that will selectively only form on one type of material on a substrate containing a pattern with different materials to create a pinning area.

### SUMMARY

One aspect of the disclosed subject matter pertains to a polymer which contain one grafting end group which has at least two hetero atoms. Specifically this polymer of structure (A) comprises two end group R₃ₚ and R₄ₚ, and a polymer chain (R) comprising either a repeat unit of structure (Ip) or a repeat unit of structure (IIp), wherein end group R₃ₚ, which derived from an anionic initiator, is either a C-1 to C-8 alkyl, a moiety of structure (IIIp), or a moiety of structure (IIIp1), but where R₃ₚ can only be selected from a C-1 to C-8 alkyl if the in the polymer chain (R) the repeat unit is (IIp), and further where R₃ₚ can only be selected from a moiety of structure (IIIp) or structure (IIIp1) if in the polymer chain (R) the repeat unit is of structure (Ip), end group R₄ₚ is either a moiety selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, a moiety of structure (IVp), a moiety of structure (IVp1), or a moiety of structure (IVp2), Further, end groups R₃ₚ and R₄ₚ cannot respectively, both simultaneously be moieties of structure (IIIp) and (IVp), both simultaneously, be moieties of structure (IIIp) and (IVp1), or both simultaneously be moieties of structure (IIIp) and (IVp2), but where said polymer of structure (A) must contain one end group moiety selected from structures (IIIp), (IVp), (IVp1) or (IVp2)

In said polymer of structure (A), in the repeat unit of structure (Ip) Rₘ₁ is a C-1 to C-8 alkyl, R₁ₚ is a C-1 to C-8 alkyl, and n1 is the number of this repeat unit in polymer chain (R).

In said polymer of structure (A), in the repeat unit of structure (IIp), Rₘ₂ is H or a C-1 to C-8 alkyl, R₂ is H or a C-1 to C-8 alkyl, n2 is the number of this repeat unit in polymer chain (R).

Further, in said polymer of structure (A), in structure (IIIp), R₁ is a chelating group, located at the para or meta position, selected from a phosphinothioic moiety of structure (Ia) an aminosulfonyl moiety of structure (Ib), a phosphonamide moiety of structure (Ic), where *** designates the attachment point of this end group moiety to the polymer of structure (A).

Further, in said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl, and * designates the attachment point of this moiety to an end group moiety of structure of (IIIp), but where said polymer of structure (A) must contain one end group moiety selected from structures (IIIp), (IVp), (IVp1) or (IVp2).

Further, in said aminosulfonyl moiety of structure (Ib), R₅ and R₆, are independently a C-1 to C-8 linear alkyl, a C-3 to C-8 branched alkyl, a C-3 to C-8 cyclic alkyl, and said dialkyl amino moiety, —N(R₉)(R₁₀), and * designates the attachment point of this moiety to an end group moiety of structure of (IIIp),

Further, in said a phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy, and * designates the attachment point of this moiety to an end group moiety of structure of (IIIp),

Further, in structure (IIIp), R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic), and R₁₅ is a C-1 to C-8 alkyl, and Rₑ₁ and Rₑ₂ are individually selected from H, a C-1 to C-8 alkyl, and a C-1 to C-8 alkoxy,

Further, in said polymer of structure (A), in structure (IIIp1) Rₑ₁ and Rₑ₂ are individually selected from H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy, and R₁₅ is a C-1 to C-8 alkyl, and *** designates the attachment point of this end group moiety to the polymer of structure (A).

Further, in said polymer of structure (A), in structure (IVp), R₁₂ is H or a C-1 to C4 alkyl, R₁₁ is a phosphinothioic moiety of structure (IIa), * * * designates the attachment point of this end group moiety to the polymer of structure (A), L₁ is a linking moiety selected from the group consisting of a direct valence bond, a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein ** designates the attachment points of the L₁ organic linking moiety to the phosphorous in structure (IIa), and * designates where L₁ within the moiety R₁₁ is attached to carbonyloxy of structure (IVp), and R₁₇ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety. In structure (IIa), * designates the attachment point of this phosphinothioic moiety to structure (IVp), R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

Further, in said polymer of structure (A), in structure (IVp1), L is a linking moiety which is either a direct valence bond or a linking group selected from a C-1 to C-8 linear alkylene, C-3 to C-8 branched alkylene, and a C-5 to C-8 cyclic alkylene, an alkyleneoxyaryl moiety (*-alkylene-O-aryl-**), and an alkylenearyl moiety (*-alkylene-aryl-**), wherein ** designates the attachment points of the L linking moiety to the phosphorous in structure (IVp1), and * designates where L is attached to said polymer of structure (A), and Rs and Rs1 are individually selected from a C-1 to C-8 alkoxy or a C-1 to C-8 alkyl and further, *** designates the attachment point of this end group moiety to the polymer of structure (A).

Further, in said polymer of structure (A), in structure (IVp2), R₁ is a chelating group, located at the para or meta position, selected from said phosphinothioic moiety of structure (Ia) said aminosulfonyl moiety of structure (Ib), and said phosphonamide moiety of structure (Ic), and R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Tb), and a phosphonamide of structure (Ic), R₁₈ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, and where *** designates the attachment point of this end group moiety to the polymer of structure (A).

Further, said polymer of structure (A) has a Mₙ ranging from about 4000 to about 7000 and a has polydispersity ranging from 1 to about 1.15.

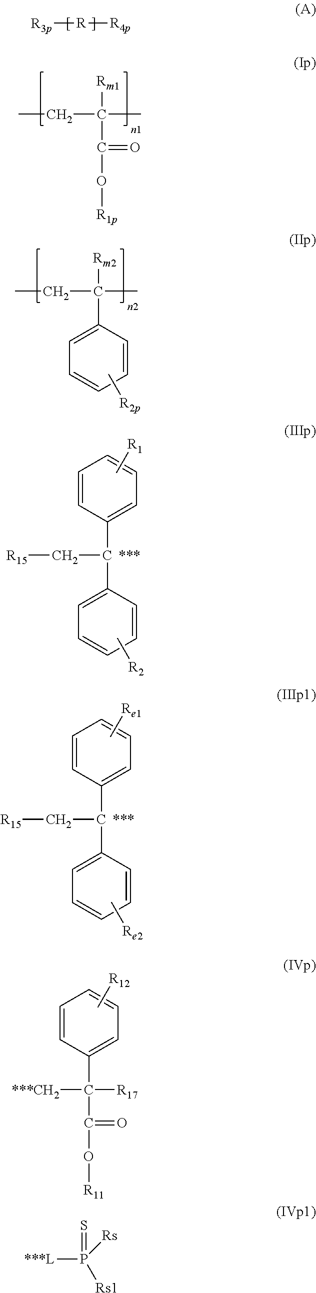

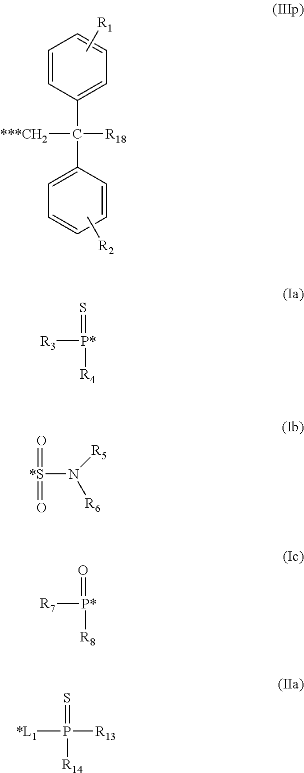

Another aspect of this invention pertains to composition of said polymer of structure (A) in an organic spin casting solvent.

Other aspects of this invention include the process of forming a pinning layer using said composition and the chemoepitaxy process of using said pinning in directed self-assembly of an overlying block copolymer and the subsequent process of etching the directed self-assembled block polymer layer into as substrate.

Yet another aspect of this invention is a novel compound of structure (I), and its use in the preparation of a polymer, wherein R₁ is a chelating group located at the meta or para position selected from a phosphinothioic moiety of structure (Ia) an aminosulfonyl moiety of structure (Ib), a phosphonamide moiety of structure (Ic) wherein * designates the attachment point of these moieties to said compound of structure (I).

In said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

In said aminosulfonyl moiety of structure (Ib), R₅ and R₆, are independently a C-1 to C-8 linear alkyl, a C-3 to C-8 branched alkyl, a C-3 to C-8 cyclic alkyl, and said dialkyl amino moiety, —N(R₉)(R₁₀).

In said phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy,

Further, in said novel compound of structure (I), R₂ is a substituent, located at the meta or para positions which is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic).

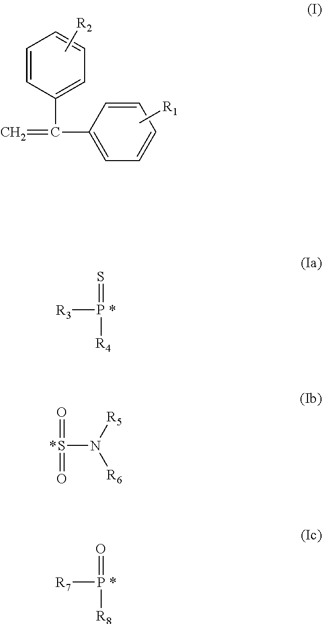

Yet another aspect of this invention is a novel compound of structure (II), and its use in the preparation of a polymer, wherein R₁₁ is a phosphinothioic moiety of structure (IIa), wherein * designates the attachment point of this moieties to said compound of structure (II), R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

Further, in this compound, L₁ is a linking moiety selected from the group consisting of a direct valence bond, a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein ** designates the attachment points of the L₁ organic linking moiety to the phosphorous in structure (IIa), and * designates where L₁ within the moiety R₁₁ is attached to the carbonyloxy of compound (II) and R₁₂ is H or a C-1 to C-4 alkyl.

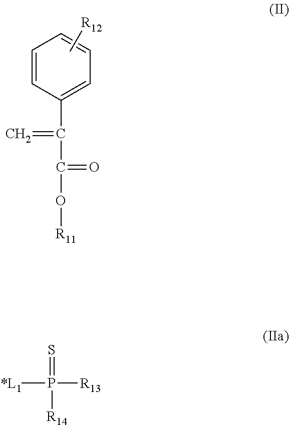

### DETAILED DESCRIPTION

It is to be understood that both the foregoing general description and the following detailed description are illustrative and explanatory and are not restrictive of the subject matter as claimed. In this application, the use of the singular includes the plural, the word “a” or “an” means “at least one,” and the use of “or” means “and/or,” unless specifically stated otherwise. Furthermore, the use of the term “including,” as well as other forms such as “includes” and “included,” is not limiting. Also, terms such as “element” or “component” encompass both elements and components comprising one unit and elements or components that comprise more than one unit, unless specifically stated otherwise. As used herein, the conjunction “and” is intended to be inclusive and the conjunction “or” is not intended to be exclusive unless otherwise indicated. For example, the phrase “or, alternatively” is intended to be exclusive. As used herein, the term “and/or” refers to any combination of the foregoing elements including using a single element.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature references and similar materials defines a term in a manner that contradicts the definition of that term in this application, this application controls.

Unless otherwise indicated, “alkyl” refers to hydrocarbon groups which can be linear, branched (e.g., methyl, ethyl, propyl, isopropyl, tert-butyl and the like) or cyclic (e.g., cyclohexyl, cyclopropyl, cyclopentyl and the like) multicyclic (e.g., norbornyl, adamantyl and the like). These alkyl moieties may be substituted or unsubstituted as described below. The term “alkyl” refers to such moieties with C-1 to C-8 carbons, unless stated otherwise. It is understood that for structural reasons linear alkyls start with C-1, while branched alkyls and cyclic alkyls start with C-3 and multicyclic alkyls start with C-5. Moreover, it is further understood that moieties derived from alkyls described below, such as alkyloxy (alkoxy), have the same carbon number ranges unless otherwise indicated. The same criteria apply to the designation C-1 to C4 alkyl. If the length of the alkyl group is specified as other than described above, the above-described definition of alkyl still stands with respect to it encompassing all types of alkyl moieties as described above and that the structural consideration with regards to minimum number of carbons for a given type of alkyl group still apply.

Alkyloxy (a.k.a. Alkoxy) refers to an alkyl group on which is attached through an oxy (—O—) moiety (e.g., methoxy, ethoxy, propoxy, butoxy, 1,2-isopropoxy, cyclopentyloxy cyclohexyloxy and the like). These alkyloxy moieties may be substituted or unsubstituted as described below. The criteria for establishing the nature of the alkyl in C-1 to C-8 alkoxy or C-1 to C4 alkoxy are the same as previously described for alkyl moieties.

Halo or halide refers to a halogen, F, Cl, Br or I which is linked by one bond to an organic moiety.

Haloalkyl refers to a linear, cyclic or branched saturated alkyl group such as defined above in which at least one of the hydrogens has been replaced by a halide selected from the group of F, Cl, Br, I or mixture of these if more than one halo moiety is present. Fluoroalkyls are a specific subgroup of these moieties.

The term “alkylene” refers to hydrocarbon groups which can be a linear, branched or cyclic which has two or more attachment points (e.g., of two attachment points: methylene, ethylene, 1,2-isopropylene, a 1,4-cyclohexylene and the like; of three attachment points 1,1,1-subsituted methane,1,1,2-subsituted ethane, 1,2,4-subsituted cyclohexane and the like). Here again, when designating a possible range of carbons, such as C-1 to C-20, as a non-limiting example, this range encompasses linear alkylenes starting with C-1 but only designates branched alkylenes, or cycloalkylene starting with C-3. These alkylene moieties may be substituted or unsubstituted as described below.

The term “aryl” or “aromatic groups” refers to such groups which contain 6 to 24 carbon atoms including phenyl, tolyl, xylyl, naphthyl, anthracyl, biphenyls, bis-phenyls, tris-phenyls and the like. These aryl groups may further be substituted with any of the appropriate substituents, e.g., alkyl, alkoxy, acyl or aryl groups mentioned hereinabove.

Unless otherwise indicated in the text, the term “substituted” when referring to an aryl, alkyl, alkyloxy, fluoroalkyl, fluoroalkyloxy, fused aromatic ring, arene, heteroarene refers to one of these moieties which also contain with one or more substituents, selected from the group of unsubstituted alkyl, substituted alkyl, unsubstituted aryl, alkyloxyaryl (alkyl-O-aryl-), dialkyloxyaryl ((alkyl-O-)₂-aryl), haloaryl, alkyloxy, alkylaryl, haloalkyl, halide, hydroxyl, cyano, nitro, acetyl, alkylcarbonyl, formyl, ethenyl (CH₂=CH—), phenylethenyl (Ph-CH═CH—), arylethenyl (Aryl-CH═CH), and substituents comprising ethenylenearylene moieties (e.g., Ar(—CH═CH—Ar—)_{z} where z is 1-3. Specific, non-limiting examples of substituted aryl and substituted aryl ethenyl substituent are as follows where “ [symbol] ” represents point of attachment;

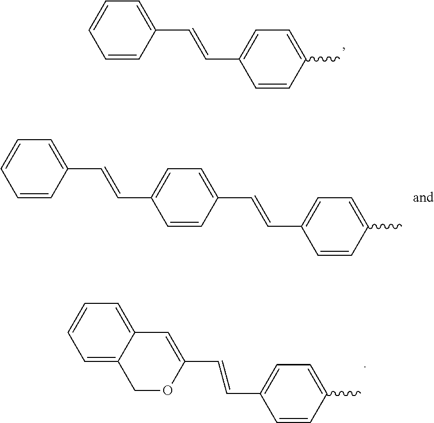

### Inventive Polymers

One aspect of this invention is a polymer of structure (A), comprising two end groups R₃ₚ and R₄ₚ, and a polymer chain (R) comprising either a repeat unit of structure (Ip) or a repeat unit of structure (Ip), wherein end group R₃ₚ, which is derived from an anionic initiator, is either a C-1 to C-8 alkyl, a moiety of structure (IIIp), or a moiety of structure (IIIp1), but where R₃ₚ can only be selected from a C-1 to C-8 alkyl if the in the polymer chain (R) the repeat unit is (IIp), and further where R₃ₚ can only be selected from a moiety of structure (IIIp) or structure (IIIp1) if in the polymer chain (R) the repeat unit is of structure (Ip). End group R₄ₚ is either a moiety selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, a moiety of structure (IVp), a moiety of structure (IVp1), or a moiety of structure (IVp2), Further, end groups R₃ₚ and R₄ₚ, cannot respectively, both simultaneously be moieties of structure (IIIp) and (IVp), both simultaneously, be moieties of structure (IIIp) and (IVp1), or both simultaneously be moieties of structure (IIIp) and (IVp2), but where said polymer of structure (A) must contain one end group moiety selected from structures (IIIp), (IVp), (IVp1) or (IVp2).

In said polymer of structure (A), in the repeat unit of structure (Ip) Rₘ₁ is a C-1 to C-8 alkyl, R₁ₚ is a C-1 to C-8 alkyl, and n1 is the number of this repeat units in polymer chain (R).

In said polymer of structure (A), in the repeat unit of structure (IIp), Rₘ₂ is H or a C-1 to C-8 alkyl, R₂ is H or a C-1 to C-8 alkyl, n2 is the number of this repeat unit in polymer chain (R).

Further, in said polymer of structure (A), in structure (IIIp), R₁ is a chelating group, located at the para or meta position, selected from a phosphinothioic moiety of structure (Ia) an aminosulfonyl moiety of structure (Ib), a phosphonamide moiety of structure (Ic), where *** designates the attachment point of this end group moiety to the polymer of structure (A).

Further, in said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl, and * designates the attachment point of this moiety to an end group moiety of structure of (IIIp).

Further, in said aminosulfonyl moiety of structure (Ib), R₅ and R₆, are independently a C-1 to C-8 linear alkyl, a C-3 to C-8 branched alkyl, a C-3 to C-8 cyclic alkyl, and said dialkyl amino moiety, —N(R₉)(R₁₀), and * designates the attachment point of this moiety to an end group moiety of structure of (IIIp).

Further, in said phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy, and * designates the attachment point of this moiety to an end group moiety of structure of (IIIp).

Further, in structure (IIIp), R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic), and R₁₅ is a C-1 to C-8 alkyl, and Rₑ₁ and Rₑ₂ are individually selected from H, a C-1 to C-8 alkyl, and a C-1 to C-8 alkoxy.

Further, in said polymer of structure (A), in structure (IIIp1) Rₑ₁ and Rₑ₂ are individually selected from H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy, and R₁₅ is a C-1 to C-8 alkyl, and *** designates the attachment point of this end group moiety to the polymer of structure (A).

Further, in said polymer of structure (A), in structure (IVp), R₁₂ is H or a C-1 to C4 alkyl, R₁₁ is a phosphinothioic moiety of structure (IIa), ** * designates the attachment point of this end group moiety to the polymer of structure (A), L₁ is a linking moiety selected from the group consisting of a direct valence bond a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein ** designates the attachment points of the L₁ organic linking moiety to the phosphorous in structure (IIa), and * designates where L₁ within the moiety R₁₁ is attached to carbonyloxy of structure (IVp), and R₁₇ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety. In structure (IIa), * designates the attachment point of this phosphinothioic moiety to structure (IVp).

R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, or a C-3 to C-8 cyclic alkyl.

Further, in said polymer of structure (A), in structure (IVp1), L is either a direct valence bond or a linking group selected from a C-1 to C-8 linear alkylene, C-3 to C-8 branched alkylene, and a C-5 to C-8 cyclic alkylene, an alkyleneoxyaryl moiety (*-alkylene-O-aryl-**), and an alkylenearyl moiety (*-alkylene-aryl-**), wherein ** designates the attachment points of the L linking moiety to the phosphorous in structure (IVp1), and * designates where L is attached to said polymer of structure (A), and Rs and Rs1 are individually selected from a C-1 to C-8 alkoxy or a C-1 to C-8 alkyl and further, *** designates the attachment point of this end group moiety to the polymer of structure (A).

Further, in said polymer of structure (A), in structure (IVp2), R₁ is a chelating group, located at the para or meta position, selected from said phosphinothioic moiety of structure (Ia) said aminosulfonyl moiety of structure (Ib), and said phosphonamide moiety of structure (Ic), and R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Tb), and a phosphonamide of structure (Ic), R₁₈ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, and where *** designates the attachment point of this end group moiety to the polymer of structure (A).

Further, said polymer of structure (A) has a Mₙ ranging from about 4000 to about 7000 and has a polydispersity ranging from 1 to about 1.15.

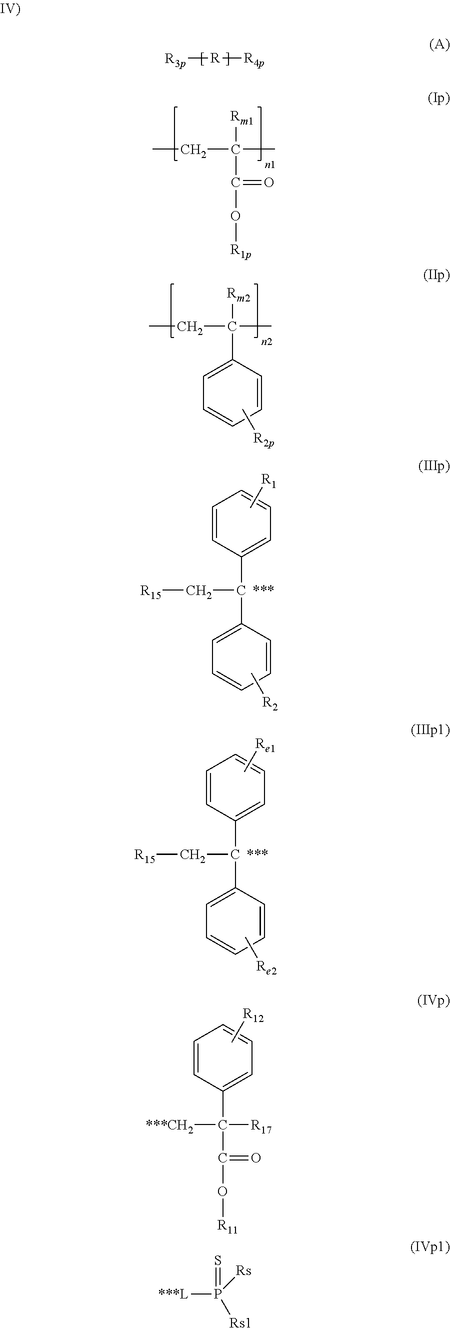

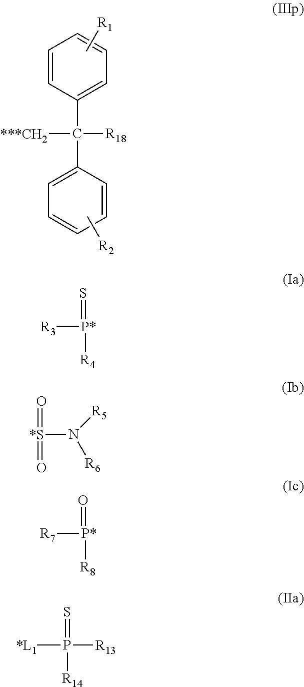

In one aspect of the inventive polymer, described herein, when the end group Rₚ it not either of the chelating moieties (IVp), (IVp1) or (IVp2) it is H. In another aspect of this embodiment, R₄ₚ is a C-1 to C-8 alkyl. In another aspect of this embodiment, Rₚ is a C-1 to C-8 alkylcarbonyl (alkyl-C(═O)—). In another aspect of this embodiment, R₄ₚ is a C-1 to C-8 trialkylsilyl ((alkyl)₃Si—). In another aspect of this embodiment, R₄ₚ is a C-1 to C-8 dialkysilyl ((alkyl)₂HSi—). In another aspect of this embodiment, R₄ₚ is a C-1 to C-8 monoalkylsilyl ((alkyl)H₂Si—). In another aspect of this embodiment, R₄ₚ is a silane (H₃Si—). In another aspect of this embodiment, R₄ₚ is a benzylic moiety.

In one aspect of the inventive polymer, when the chelating moiety (IVp) is present R₁₇ is H. In another aspect of this embodiment, R₁₇ is a C-1 to C-8 alkyl. In another aspect of this embodiment, In another aspect of this embodiment, R₁₇ is a C-1 to C-8 alkylcarbonyl (alkyl-C(═O)—). In another aspect of this embodiment, R₁₇ is a C-1 to C-8 trialkylsilyl ((alkyl)₃Si—). In another aspect of this embodiment, R₁₇ is a C-1 to C-8 dialkysilyl ((alkyl)₂HSi—). In another aspect of this embodiment, R₁₇ is a C-1 to C-8 monoalkylsilyl ((alkyl)H₂Si—). In another aspect of this embodiment, R₁₇ is a silane (H₃Si—). In another aspect of this embodiment, R₁₇ is a benzylic moiety.

In one aspect of the inventive polymer, when the chelating moiety (IVp2) is present, R₁₈ is H. In another aspect of this embodiment, R₁₈ is a C-1 to C-8 alkyl. In another aspect of this embodiment, R₁₈ is a C-1 to C-8 alkylcarbonyl (alkyl-C(═O)—). In another aspect of this embodiment, R₁₈ is a C-1 to C-8 trialkylsilyl ((alkyl)₃Si—). In another aspect of this embodiment, R₁₈ is a C-1 to C-8 dialkysilyl ((alkyl)₂HSi—). In another aspect of this embodiment, R₁₈ is a C-1 to C-8 monoalkylsilyl ((alkyl)H₂Si—). In another aspect of this embodiment, R₁₈ is a silane (H₃Si—). In another aspect of this embodiment, R₁₈ is a benzylic moiety.

In one aspect of the inventive polymer of structure (A), it has the more specific structure (A-1).

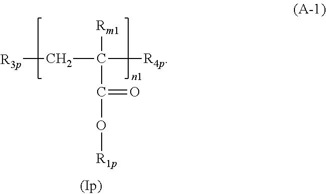

In one aspect of the inventive polymer of structure (A-1), it has structure (A-2). In one aspect of this embodiment Rₘ₁ is methyl. In another aspect of these embodiments, R₁ₚ is methyl.

In yet another aspect of these embodiments, R₂ is H.

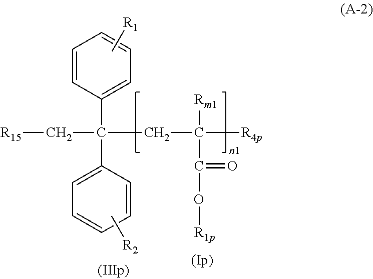

In another aspect of the inventive polymer of structures (A-1) or (A-2), it has structure (A-2a). In another embodiment of this structure, it has structure (A-2b). In one aspect of these embodiments, R₃ and R₄ are independently selected from a C-1 to C4 alkoxy or a C-1 to C4 alkyl. In another aspect of these embodiments, R₃ and R₄ are independently selected from a C-1 to C4 alkoxy. In another aspect of these embodiments, R₃ and R₄ are independently selected from a C-1 to C4 alkyl. In another aspect of these embodiments, R₃ and R₄ are both ethoxy. In another aspect of these embodiments, R₃ and R₄ are both methoxy. In yet another embodiment of this structure it has structure (A-2c). In one aspect of these embodiments, Rₘ₁ is methyl. In another aspect of these embodiments, R₁ₚ is methyl. In yet another aspect of these embodiments, R₂ is H.

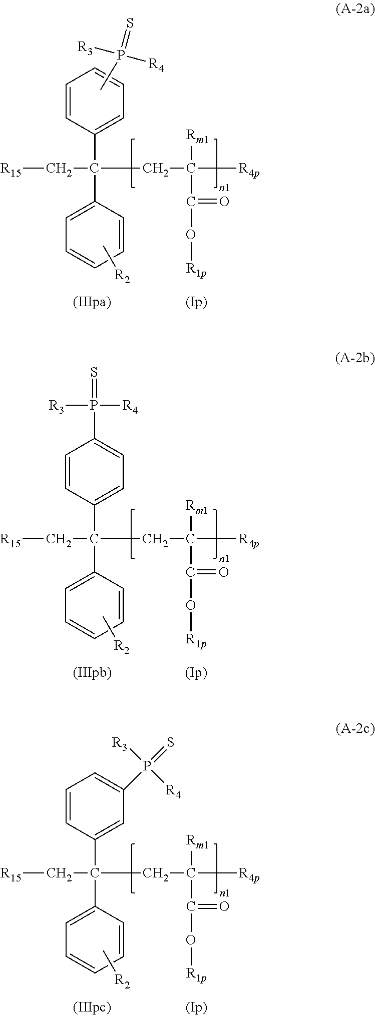

In another aspect of the inventive polymer of structures (A-1) or (A-2), it has structure (A-2d). In another aspect of this embodiment, it has structure (A-2e). In yet another embodiment of this structure it has structure (A-2f). In one aspect of these embodiments, R₅ and R₆ are independently selected from a C-1 to C4 alkyl. In another aspect of these embodiments, R₅ and R₆ are independently selected from a C-1 to C-2 alkyl. In one aspect of these embodiments, R₅ and R₆ are methyl. In one aspect of these embodiments, Rₘ₁ is methyl. In another aspect of these embodiments, R₁ₚ is methyl. In yet another aspect of these embodiments, R₂ is H.

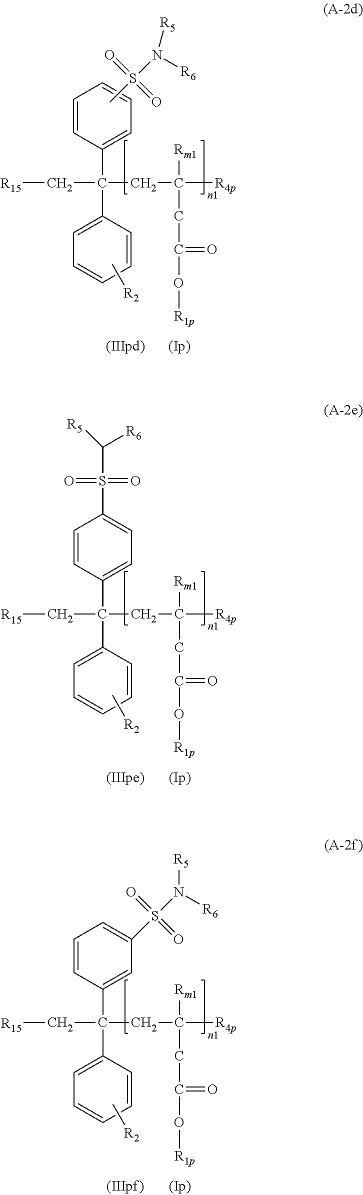

In another aspect of the inventive polymer of structures (A-1) or (A-2) it has structure (A-2g). In another embodiment of this structure, it has structure (A-2h). In yet another aspect of this embodiment it has structure (A-2i). In one aspect of these embodiments, R₉ and R₁₀ are independently selected from a C-1 to C4 alkyl. In another aspect of these embodiments, R₉ and R₁₀ are independently selected from a C-1 to C-2 alkyl. In one aspect of these embodiments, R₉ and R₁₀ are methyl. In one aspect of the embodiments where R₉ and R₁₀ are independently selected from a C-1 to C4 alkyl, R₈ is selected from a C-1 to C4 alkyl or an aryl; in one aspect of this embodiment R₈ is a C-1 to C-2 alkyl; in another aspect of this embodiment said aryl is phenyl. In one aspect of these embodiments, Rₘ₁ is methyl. In another aspect of these embodiments, R₁ₚ is methyl. In et another aspect of these embodiments R₂ is H.

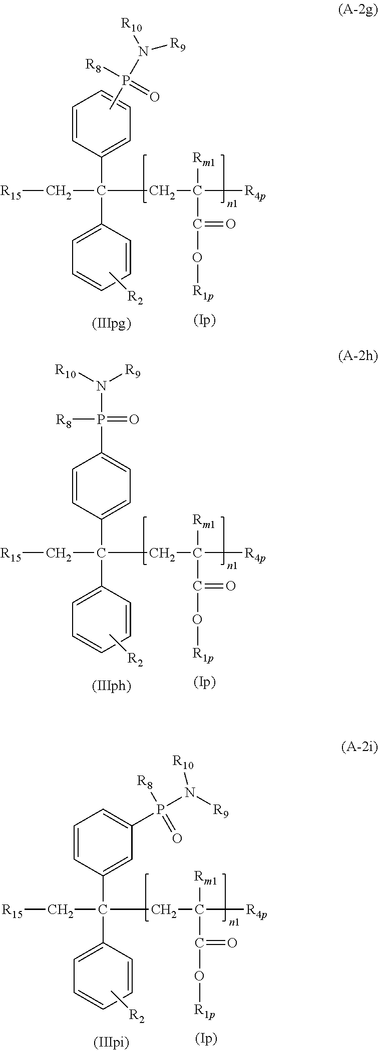

In another aspect of the inventive polymer of structures (A-1) or (A-2) it has structure (A-3). In one aspect of this polymer L₁ is a direct valence bond. In another aspect of this polymer L₁ is a C-2 to C-8 alkylene moiety. In another aspect of this polymer wherein L₁ is an arylene moiety (-aryl-). In yet another aspect of this polymer L₁ is an alkyleneoxyaryl moiety (*-alkylene-O-aryl-**). In another aspect of these embodiments, R₁₃ and R₁₄ are independently selected from a C-1 to C4 alkyl and a C-1 to C4 alkoxy. In another aspect of these embodiments, R₁₃ and R₁₄ are independently selected from a C-1 to C4 alkoxy. In another aspect of these embodiments, R₁₃ and R₁₄ are ethoxy. In another aspect of these embodiments, R₁₃ and R₁₄ are methoxy. In another aspect of these embodiments, R₁₃ and R₁₄ are independently selected from a C-1 to C4 alkyl. In another aspect of these embodiments, R₁₃ and R₁₄ are ethyl. In another aspect of these embodiments, R₁₃ and R₁₄ are ethyl. In one aspect of these embodiments, Rₘ₁ is methyl. In another aspect of these embodiments, Rip is methyl. In yet another aspect of these embodiments, Rₑ₁ and Rₑ₂ are both H. In another aspect of these embodiments, R₁₇ is H. In another aspect of these embodiments, R₁₂ is H.

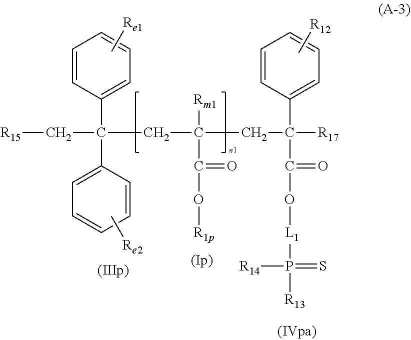

In another aspect of the polymer of structure (A-3), where L₁ is a C-2 to C-8 alkylene moiety it has structure (A-3a) or structure (A-3b) where and n is an integer ranging from 1 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl. In another aspect of this embodiment R₁₃ and R₁₄ are selected from a C-1 to C4 alkoxy. In another aspect of this embodiment R₁₃ is selected from a C-1 to C4 alkoxy and R₁₄ is selected from a C-1 to C4 alkyl. In one aspect of these embodiments, Rₘ₁ is methyl. In another aspect of these embodiments, R₁ₚ is methyl. In yet another aspect of these embodiments, Rₑ₁ and Rₑ₂ are both H. In another aspect of these embodiments, R₁₇ is H. In another aspect of these embodiments, R₁₂ is H.

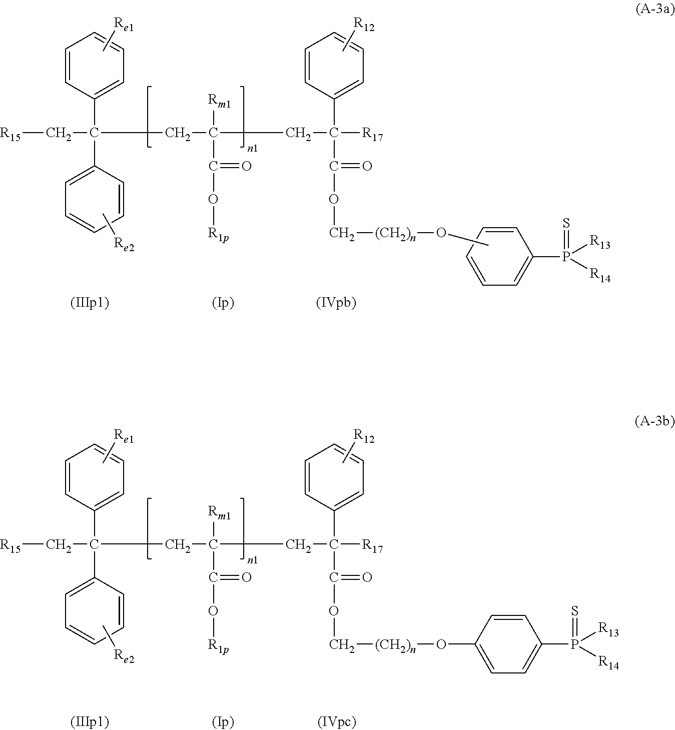

In another aspect of the polymer of structure (A-3) it has structures (A-3c) or (A-3d), wherein L₁ is alkylenearyl moiety (*-alkylene-aryl-**) where n′ is an integer ranging from 0 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl, In another aspect of this embodiment R₁₃ and R₁₄ are selected from a C-1 to C4 alkoxy. In another aspect of these embodiments, R₁₃ is selected from a C-1 to C4 alkoxy and R₁₄ is selected from a C-1 to C4 alkyl. In one aspect of these embodiments, Rₘ₁ is methyl. In another aspect of these embodiments, R₁ₚ is methyl. In yet another aspect of these embodiments, Rei and Rₑ₂ are both H. In another aspect of these embodiments, R₁₇ is H. In another aspect of these embodiments, R₁₂ is H.

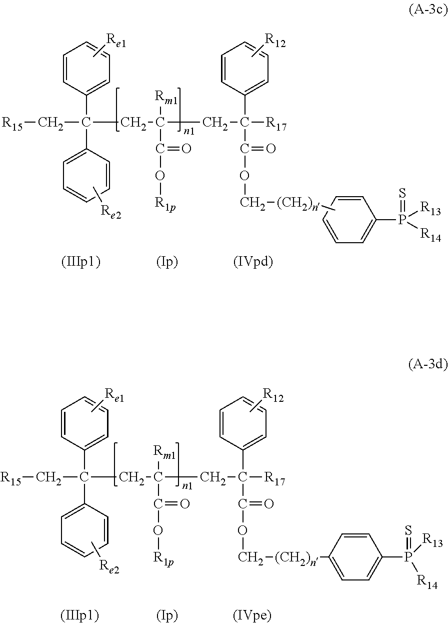

In another aspect of the inventive polymer of structures (A-1) or (A-2) it has structure (A-4). In one aspect of this embodiment, L is a direct valence bond. In another aspect of this embodiment L is a C-2 to C-8 alkylene moiety. In another aspect of this embodiment L is an arylene moiety (-aryl-). In yet another aspect of this embodiment L is an alkyleneoxyaryl moiety (*-alkylene-O-aryl-**). In still another aspect of this embodiment L is a an alkylenearyl moiety (*-alkylene-aryl-**). In one aspect of these embodiments, Rₘ₁ is methyl. In another aspect of these embodiments, R₁ₚ is methyl. In yet another aspect of these embodiments, Rₑ₁ and Rₑ₂ are both H.

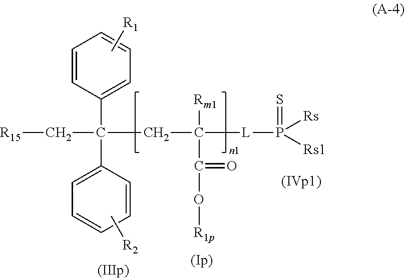

In another aspect of the inventive polymer of structures (A-4) it has structure (A-4a) or structure (A-4b), wherein n′ is an integer ranging from 0 to 7. In another aspect of this embodiment, it has structure (A-4b). In another aspect of these embodiments, Rs and Rs1 are individually selected from a C-1 to C4 alkyl. In another aspect of these embodiments, Rs and Rs1 are individually selected from a C-1 to C-4 alkoxy. In one aspect of these embodiments, Rₘ₁ is methyl. In another aspect of these embodiments, R₁ₚ is methyl. In yet another aspect of these embodiments, Rₑ₁ and Rₑ₂ are both H.

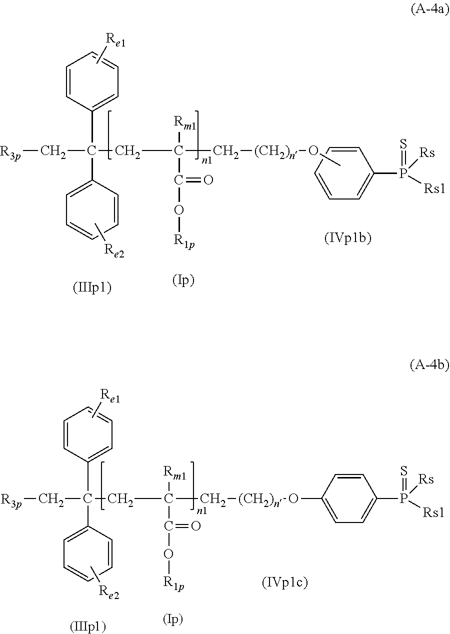

In another aspect of the inventive polymer of structures (A-4) it has structure (A-4c) or structure (A-4d), wherein n′ is an integer ranging from 0 to 7. In another aspect of this embodiment, it has structure (A-4d). In another aspect of these embodiments, Rs and Rs1 are individually selected from a C-1 to C-4 alkyl. In another aspect of these embodiments, Rs and Rs1 are individually selected from a C-1 to C-4 alkoxy. In one aspect of these embodiments, Rₘ₁ is methyl. In another aspect of these embodiments, R₁ₚ is methyl. In yet another aspect of these embodiments, Rₑ₁ and Rₑ₂ are both H.

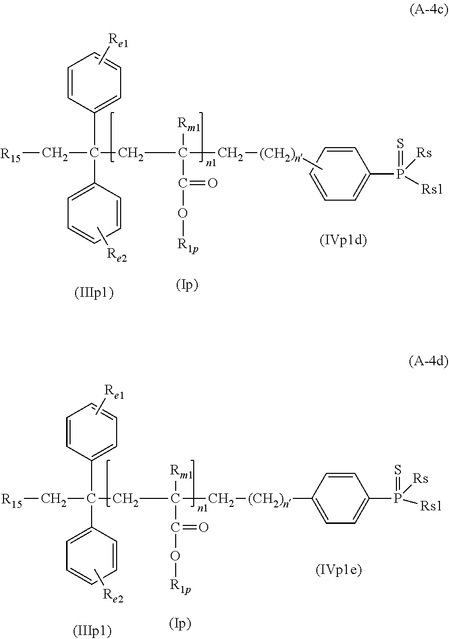

In one aspect of the inventive polymer of structure (A), it has the more specific structure (B-1), wherein R₃ₚ is a C-1 to C-8 alkyl.

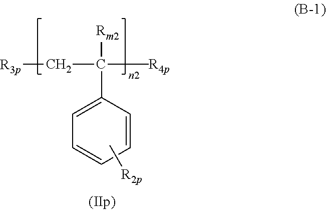

In another aspect of the inventive polymer of structures (B-1) it has structure (B-1a), wherein R₃ₚ is a C-1 to C-8 alkyl. In another aspect of R₃ₚ is a C-1 to C-8 alkyl, and L₁ is a direct valence bond. In another aspect of R₃ₚ is a C-1 to C-8 alkyl, and L₁ is a C-2 to C-8 alkylene moiety. In another aspect of this embodiment R₃ₚ is a C-1 to C-8 alkyl, and L₁ is an arylene moiety (-aryl-). In another aspect of this embodiment R₃ₚ is a C-1 to C-8 alkyl, and L₁ is an alkyleneoxyaryl moiety (*-alkylene-O-aryl-**). In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl. In another aspect of this embodiment R₁₃ and R₁₄ are selected from a C-1 to C4 alkoxy. In another aspect of these embodiments, R₁₃ is selected from a C-1 to C4 alkoxy and R₁₄ is selected from a C-1 to C4 alkyl.

In another aspect of these embodiments, Rₘ₂ is H. In yet another aspect of these embodiments, R₂ₚ is H.

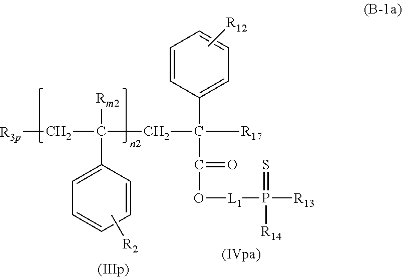

In one aspect of the inventive polymer of structure (B-1) it has structures (B-1b) or (B1c) wherein R₃ₚ is a C-1 to C-8 alkyl and n is an integer ranging from 1 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl, In another aspect of this embodiment R₁₃ and R₁₄ are selected from a C-1 to C4 alkoxy. In another aspect of these embodiments, R₁₃ is selected from a C-1 to C4 alkoxy and R₁₄ is selected from a C-1 to C-4 alkyl. In another aspect of these embodiments, Rₘ₂ is H. In yet another aspect of these embodiments, R₂ₚ is H. In another aspect of these embodiments, R₁₇ is H. In another aspect of these embodiments, R₁₂ is H.

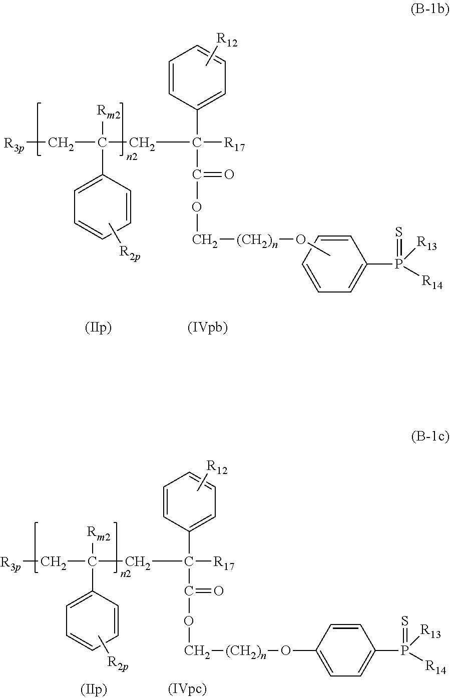

In one aspect of the inventive polymer of structure (B-1), it has the more specific structures (B-1d) or (B-1e), wherein R₃ₚ is a C-1 to C-8 alkyl and n′ is an integer ranging from 0 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to CA alkyl or alkoxy. In another aspect of this embodiment R₁₃ and R₁₄ are selected from a C-1 to CA alkyl. In another aspect of this embodiment, R₁₃ and R₁₄ are selected from a C-1 to C-4 alkoxy. In another aspect of these embodiments, R₁₃ is selected from a C-1 to C-4 alkoxy and R₁₄ is selected from a C-1 to C-4 alkyl. In another aspect of these embodiments, Rₘ₂ is H. In yet another aspect of these embodiments, R₂ₚ is H. In another aspect of these embodiments, R₁₇ is H. In another aspect of these embodiments, R₁₂ is H.

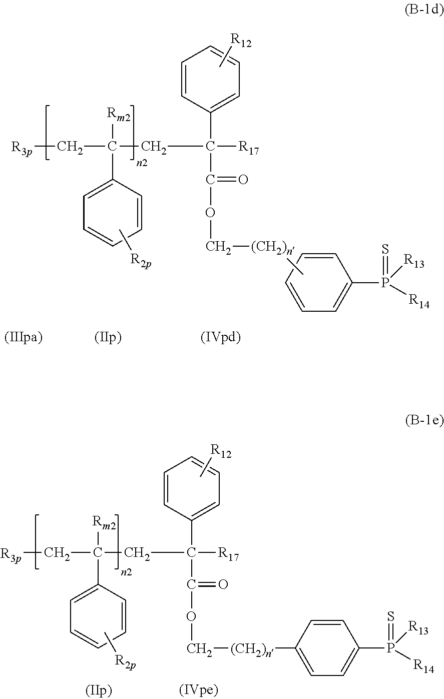

In another aspect of the inventive polymer of structure (B-1) it has structure (B-2). In one aspect of this embodiment L is a direct valence bond. In another aspect of this embodiment L is a C-2 to C-8 alkylene moiety. In another aspect of this embodiment wherein L is an arylene moiety (-aryl-). In another aspect of this embodiment, is an alkyleneoxyaryl moiety (*-alkylene-O-aryl-**). In another aspect of this embodiment is where Rs and Rs1 are individually selected from a C-1 to C-4 alkyl. In another aspect of this embodiment Rs and Rs1 are individually selected from a C-1 to C-4 alkoxy. In yet another embodiment Rs and Rs1 are methyl. In another aspect of this embodiment Rs and Rs1 are methoxy. In another aspect of these embodiments, Rₘ₂ is H. In yet another aspect of these embodiments, R₂ₚ is H.

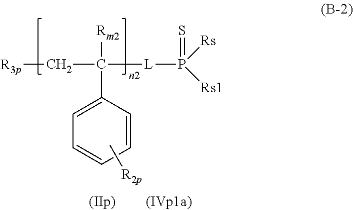

In the aspect of the inventive polymer of structure (B-2), where L is a direct valence bond and it has structure (B-2aa), another aspect of this embodiment is where Rs and Rs1 are individually selected from a C-1 to C4 alkyl. In another aspect of this embodiment Rs and Rs1 are individually selected from a C-1 to C4 alkoxy. In yet another embodiment Rs and Rs1 are methyl. In another aspect of this embodiment Rs and Rs1 are methoxy. In another aspect of these embodiments, Rₘ₂ is H. In yet another aspect of these embodiments, R₂ₚ is H.

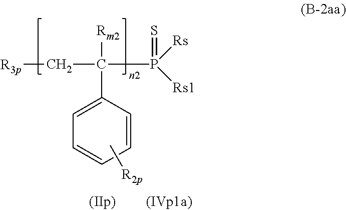

In another aspect of the said polymer of structure (B-2) it has structure (B-2a) or structure (B-2b) where R₁₅ is a C-1 to C-8 alkyl, and n′ is an integer ranging from 0 to 7. In one aspect of these embodiments, Rs and Rs1 are independently selected from a C-1 to C-8 alkyl or alkoxy. In another aspect of these embodiments, Rs and Rs1 are individually selected from a C-1 to C4 alkyl. In another aspect of these embodiments, Rs and Rs1 are individually selected from a C-1 to C4 alkoxy. In yet another embodiment Rs and Rs1 are methyl. In another aspect Rs and Rs1 are methoxy. In another aspect of these embodiments, Rₘ₂ is H. In yet another aspect of these embodiments, R₂ₚ is H.

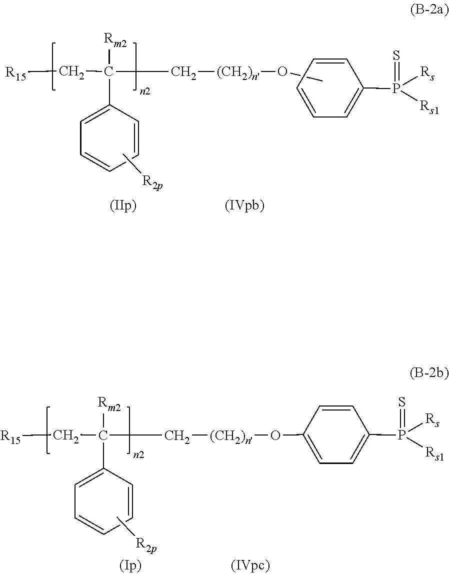

In another aspect of the said polymer of structure (B-2) it has structure (B-2c) or structure (B-2d), where R₁₅ is a C-1 to C-8 alkyl, and n′ is an integer ranging from 0 to 7. In one aspect of these embodiments, Rs and Rs1 are independently selected from a C-1 to C-8 alkyl or alkoxy. In another aspect of these embodiments, Rs and Rs1 are individually selected from a C-1 to C-4 alkyl. In another aspect of these embodiments, Rs and Rs1 are individually selected from a C-1 to C-4 alkoxy. In yet another embodiment Rs and Rs1 are methyl. In another aspect Rs and Rs1 are methoxy. In another aspect of these embodiments, Rₘ₂ is H. In yet another aspect of these embodiments, R₂ₚ is H.

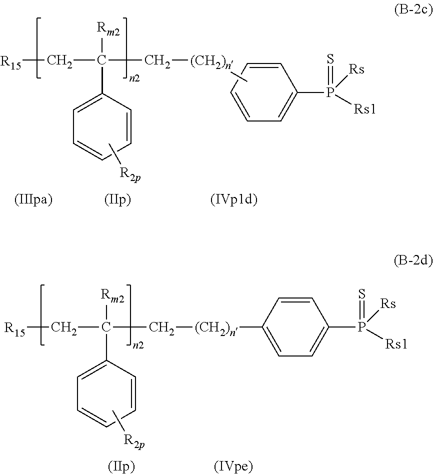

In another aspect of the inventive polymer of structures (B-1) it has structure (B-3). In one aspect of these embodiments, R₃ and R₄ are independently selected from a C-1 to C-4 alkoxy or a C-1 to C-4 alkyl. In one aspect of this embodiment R₂ₚ is H. In another aspect of this embodiment Rₘ₂ is H. In another aspect of this embodiment R₂ is H. In another aspect of these embodiments, R₁₈ is H.

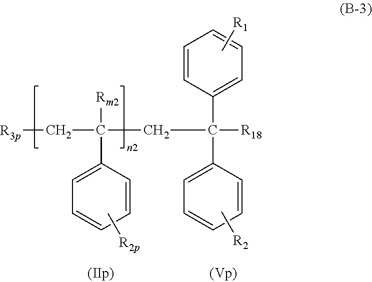

In another aspect of the said polymer of structure (B-3) it has structure (B-3a), structure (B-3b), or structure (B-3c). In one aspect of these embodiments, R₃ and R₄ are independently selected from a C-1 to C4 alkoxy or a C-1 to C4 alkyl. In another aspect of these embodiments, R₃ and R₄ are independently selected from a C-1 to C4 alkoxy. In another aspect of these embodiments, R₃ and R₄ are independently selected from a C-1 to C4 alkyl. In another aspect of these embodiments, R₃ and R₄ are both ethoxy. In another aspect of these embodiments, R₃ and R₄ are both methoxy. In one aspect of these embodiments, R₂, is H. In another aspect of these embodiments, Rₘ₂ is H. In another aspect of these embodiments, R₂ is H. In another aspect of these embodiments, R₁₈ is H.

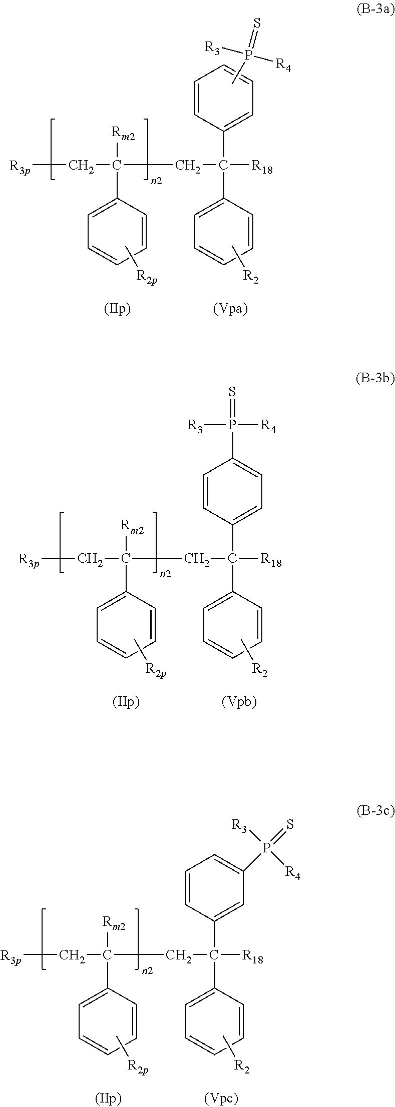

In another aspect of the said polymer of structure (B-3), it has structure (B-3d), structure (B-3e) or structure (B-3f). In one aspect of these embodiments, R₅ and R₆ are independently selected from a C-1 to C4 alkyl. In another aspect of these embodiments, R₅ and R₆ are independently selected from a C-1 to C-2 alkyl. In one aspect of these embodiments, R₅ and R₆ are methyl. In one aspect of these embodiments, R₂ₚ is H. In another aspect of these embodiments, Rₘ₂ is H. In another aspect of these embodiments, R₂ is H. In another aspect of these embodiments, R₁₈ is H.

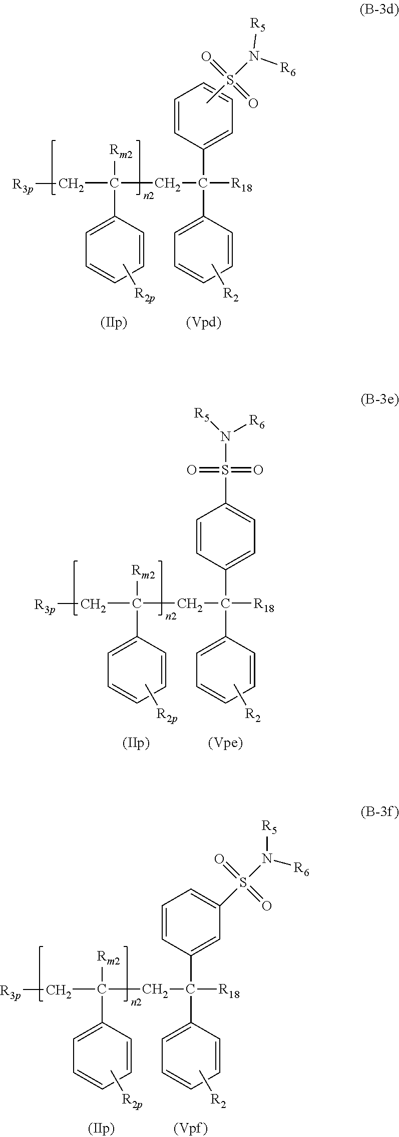

In another aspect of the said polymer of structure (B-3), it has structure (B-3g), structure (B-3h) or structure (B-3i). In one aspect of these embodiments, R₉ and R₁₀ are independently selected from a C-1 to C4 alkyl. In another aspect of these embodiments, R₉ and R₁₀ are independently selected from a C-1 to C-2 alkyl. In one aspect of these embodiments, R₉ and R₁₀ are methyl. In one aspect of the embodiments where R₉ and R₁₀ are independently selected from a C-1 to C4 alkyl, Rs is selected from a C-1 to C4 alkyl or an aryl; in one aspect of this embodiment Rs is a C-1 to C-2 alkyl; in another aspect of this embodiment said aryl is phenyl. In one aspect of these embodiments, R₂ₚ is H. In another aspect of these embodiments, Rₘ₂ is H. In another aspect of these embodiments, R₂ is H. In another aspect of these embodiments, R₁₈ is H.

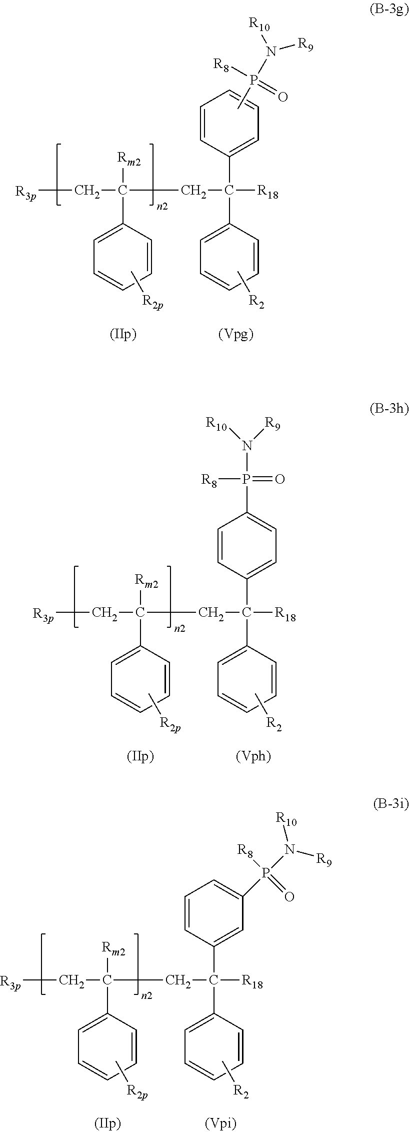

### Inventive Compositions

Another aspect of this invention are compositions of any one of the inventive polymers described herein having structure (A) and an organic spin casting solvent.

Another aspect of this invention are compositions of any one of the inventive polymers described herein having structure (A-1) and an organic spin casting solvent.

Another aspect of this invention are compositions comprising of any one of the inventive polymers described herein having any one of structures (A-2), (A-2a), (A-2b), (A-2c), (A-2d), (A-2e), (A-2f), (A-2g), (A-2h), or (A-2i), and an organic spin casting solvent. In one aspect of this embodiment, said polymer has structure (A-2). In one aspect of this embodiment, said polymer has structure (A-2a). In one aspect of this embodiment, said polymer has structure (A-2b). In one aspect of this embodiment, said polymer has structure (A-2c). In one aspect of this embodiment, said polymer has structure (A-2d). In one aspect of this embodiment, said polymer has structure (A-2e). In one aspect of this embodiment, said polymer has structure (A-2f). In one aspect of this embodiment, said polymer has structure (A-2g). In one aspect of this embodiment, said polymer has structure (A-2h). In one aspect of this embodiment, said polymer has structure (A-2i). In another aspect of this embodiment said composition consists of any one of the above-described polymer structures and an organic spin casting solvent.

Another aspect of this invention are compositions comprising of any one of the inventive polymers described herein having any one of structures (A-3), (A-3a), (A-3b), (A-3c), or (A-3d) and an organic spin casting solvent. In one aspect of this embodiment, said polymer has structure (A-3). In one aspect of this embodiment, said polymer has structure (A-3a). In one aspect of this embodiment, said polymer has structure (A-3b). In one aspect of this embodiment, said polymer has structure (A-3c). In one aspect of this embodiment, said polymer has structure (A-3d). In another aspect of this embodiment said composition consists only of any one the polymer structures described in this embodiment and an organic spin casting solvent.

Another aspect of this invention are compositions comprising of any one of the inventive polymers described herein having any one of structures (A-4), (A-4a), (A-4b), (A-4c), or (A-4d) and an organic spin casting solvent. In one aspect of this embodiment, said polymer has structure (A-4). In one aspect of this embodiment, said polymer has structure (A-4a). In one aspect of this embodiment, said polymer has structure (A-4b). In one aspect of this embodiment, said polymer has structure (A-4c). In one aspect of this embodiment, said polymer has structure (A-4d). In another aspect of this embodiment said composition consists only of any one the polymer structures described in this embodiment and an organic spin casting solvent.

Another aspect of this invention are compositions comprising of any one of the inventive polymers described herein having any one of structures (B-1), (B-1a), (B-1b), (B-1c), (B-1d), or (B-1e). In one aspect of this embodiment, said polymer has structure (B-1). In one aspect of this embodiment, said polymer has structure (B-1a). In one aspect of this embodiment, said polymer has structure (B-1b). In one aspect of this embodiment, said polymer has structure (B-1c). In one aspect of this embodiment, said polymer has structure (B-1d). In one aspect of this embodiment, said polymer has structure (B-1e). In another aspect of this embodiment said composition consists only of any one the polymer structures described in this embodiment and an organic spin casting solvent.

Another aspect of this invention are compositions comprising of any one of the inventive polymers described herein having any one of structures (B-2), (B-2a), (B-2b), (B-2c), or (B-2d). In one aspect of this embodiment, said polymer has structure (B-2). In one aspect of this embodiment, said polymer has structure (B-2a). In one aspect of this embodiment, said polymer has structure (B-2b). In one aspect of this embodiment, said polymer has structure (B-2c). In one aspect of this embodiment, said polymer has structure (B-2d). In another aspect of this embodiment said composition consists only of any one the polymer structures described in this embodiment and an organic spin casting solvent.

Another aspect of this invention are compositions comprising of any one of the inventive polymers described herein having any one of structures (B-3), (B-3a), (B-3b), (B-3c), (B-3d), (B-3e), (B-3f), (B-3g), (B-3h) and (B-3i). In one aspect of this embodiment, said polymer has structure (B-2). In one aspect of this embodiment, said polymer has structure (B-2a). In one aspect of this embodiment, said polymer has structure (B-2b). In one aspect of this embodiment, said polymer has structure (B-2c). In one aspect of this embodiment, said polymer has structure (B-2d). In another aspect of this embodiment said composition consists only of any one the polymer structures described in this embodiment and an organic spin casting solvent.

In the above embodiments of the novel compositions, the organic spin casting solvent is one which can dissolve said novel polymers and any other additional optional components as noted above. This organic spin casting solvent may be a single solvent or a mixture of solvents. Suitable solvents are organic solvent which may include, for example, a glycol ether derivative such as ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA); carboxylates such as ethyl acetate, n-butyl acetate and amyl acetate; carboxylates of di-basic acids such as diethyloxylate and diethylmalonate; dicarboxylates of glycols such as ethylene glycol diacetate and propylene glycol diacetate; and hydroxy carboxylates such as methyl lactate, ethyl lactate (EL), ethyl glycolate, and ethyl-3-hydroxy propionate; a ketone ester such as methyl pyruvate or ethyl pyruvate; an alkyloxycarboxylic acid ester such as methyl 3-methoxypropionate, ethyl 3-ethoxypropionate, ethyl 2-hydroxy-2-methylpropionate, or methylethoxypropionate; a ketone derivative such as methyl ethyl ketone, acetyl acetone, cyclopentanone, cyclohexanone or 2-heptanone; a ketone ether derivative such as diacetone alcohol methyl ether; a ketone alcohol derivative such as acetol or diacetone alcohol; a ketal or acetal like 1,3 dioxalane and diethoxypropane; lactones such as butyrolactone; an amide derivative such as dimethylacetamide or dimethylformamide, anisole, and mixtures thereof.

The novel compositions, in addition to the polymer and the solvent, may contain surfactants as additives to facilitate coating.

Another aspect of said invention is novel compositions wherein said novel polymers comprise from about 0.1 wt. % to about 10 wt. % of the total weight of said composition including the organic spin casting solvent. In another aspect it comprises from about 0.1 wt. % to about 2 wt. %. In yet another embodiment it comprises from about 0.5 wt % to about 1.5 wt %. In yet another embodiment it comprises from about 0.75 wt. % to about 1.5 wt. %. In yet another embodiment it comprises about 1.0 wt. %.

### Processes of Using Inventive Compositions

Another aspect of this invention is a process of forming a pinning layer brush selectively on a substrate which comprises both metallic surface areas and non-metallic surface areas, comprising the steps;
i) coating the composition comprising any one of the polymer having structure (A), or its substructures on a said substrate forming a film,
ii) baking said film at a temperature from about 120° C. to about 250° C. for about 1 minute to about 1 hour to form a baked film,
iii) washing said baked film with a solvent to remove ungrafted polymer forming a pinning layer brush only on said metallic surface areas of said substrate.
   In one aspect of this embodiment n said metallic surface areas are selected from the group consisting of Cu, Au, Ag, W, Ta, Nb, Fe, Ni, Co, Mo, Al, Pt, Rh, Pb, Cd, Ti, Zr, Hf, and Ru and said non-metallic surface areas are selected from the group consisting of Si, Silicon oxide (SiOx), Silicon nitride (SiNx), Silicon oxynitride (SiON) and organic dielectric substrates.

Another aspect of this invention is a process comprising the steps;
ia) coating a composition comprising any one of the polymer having structure (A), or its substructures, on a substrate which comprises both metallic surface areas and non-metallic surface areas forming a film,
iia) baking said film at a temperature from about 120° C. to about 250° C. for about 1 minute to about 1 hour to form a baked film,
iiia) washing said baked film with a solvent to remove ungrafted polymer forming a grafted substrate wherein pinning layer brush are only present on said metallic surface areas of said substrate,
iva) coating said grafted substrate with a neutral layer composition forming a neutral layer coating,
va) curing said neutral layer coating,
via) washing away, with a solvent, uncured neutral layer, leaving in said non-metallic areas a neutral directing brush, forming on said substrate a chemoepitaxy directing layer,
viia) coating on said chemoepitaxy directing layer with a block copolymer solution forming a coating of block copolymer,
viiia) annealing said coating of block copolymer to form a directed self-assembled film of the block copolymer on said chemoepitaxy directing layer.
   In one aspect of this process in said substrate, said metallic surface areas are Tungsten and said non-metallic surface areas are Silicon or Silicon oxide. In another aspect of this said block copolymer is a block copolymer comprised of styrenic repeat units and alkyl acrylic repeat units.
   In another aspect of this embodiment said said block copolymer is either an AB diblock copolymer of alkyl acrylic repeat unit and styrenic repeat units, or an ABA triblock copolymer of alkyl acrylic repeat unit and styrenic repeat units.

### Inventive Compounds

Another aspect of this invention are novel compounds which are useful in the synthesis of the inventive polymers of structure (A) and its substructures as described herein.

One embodiment of these novel compounds is those having structure (I), wherein R₁ is a chelating group located at the meta or para position selected from a phosphinothioic moiety of structure (Ia) an aminosulfonyl moiety of structure (Ib), a phosphonamide moiety of structure (Ic) wherein * designates the attachment point of these moieties to said compound of structure (I).

Further, in said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

Further, in said aminosulfonyl moiety of structure (Ib), R₅ and R₆, are independently a C-1 to C-8 linear alkyl, a C-3 to C-8 branched alkyl, a C-3 to C-8 cyclic alkyl, and dialkyl amino moiety, —N(R₉)(R₁₀).

Further, in said phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy. In one aspect of this embodiment Rs is an aryl, and in a more specific aspect of this embodiment is phenyl. In one aspect of this embodiment Rs is an alkylenearyl. In one aspect of this embodiment R₈ is a C-2 to C-8 alkyleneoxyalkyl. In one aspect of this embodiment R₈ is a C-2 to C-8 haloalkyl. In one aspect of this embodiment R₈ is a C-1 to C-8 linear alkyl. In one aspect of this embodiment R₈ is a C-3 to C-8 branched alkyl. In one aspect of this embodiment R₈ is a C-3 to C-8 cyclic alkyl. In one aspect of this embodiment R₈ is a C-1 to C-8 linear alkyloxy. In one aspect of this embodiment Rs is a C-3 to C-8 branched alkyloxy. In one aspect of this embodiment R₈ is or a C-3 to C-8 cyclic alkyloxy.

Further, in said compound of structure (I), R₂ is a substituent, located at the meta or para positions which is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic).

Specifically, when both R₁ and R₂ are a phosphonamide moiety of structure (Ic), R₈ may individually be selected form the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy. In one aspect of this embodiment R₈ is an aryl, and in a more specific aspect of this embodiment is phenyl. In one aspect of this embodiment R₈ is an alkylenearyl. In one aspect of this embodiment R₈ is a C-2 to C-8 alkyleneoxyalkyl. In one aspect of this embodiment R₈ is a C-2 to C-8 haloalkyl. In one aspect of this embodiment R₈ is a C-1 to C-8 linear alkyl. In one aspect of this embodiment R₈ is a C-3 to C-8 branched alkyl. In one aspect of this embodiment R₈ is a C-3 to C-8 cyclic alkyl. In one aspect of this embodiment R₈ is a C-1 to C-8 linear alkyloxy. In one aspect of this embodiment Rs is a C-3 to C-8 branched alkyloxy. In one aspect of this embodiment R₈ is or a C-3 to C-8 cyclic alkyloxy.

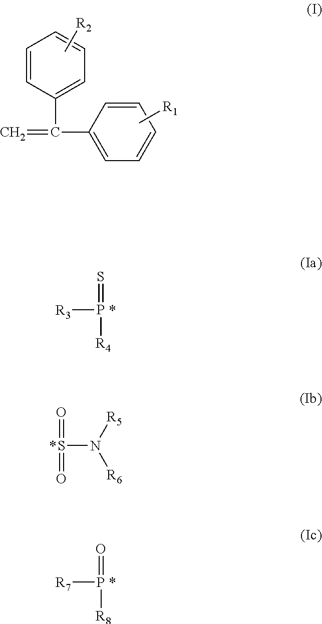

In another embodiment of the compound of structure (I) it has the more specific structure (I-1).

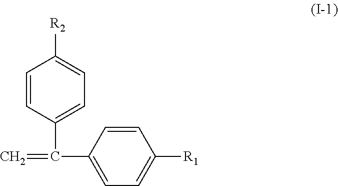

In another embodiment of the compound of structure (I), it has the more specific structure (I-2).

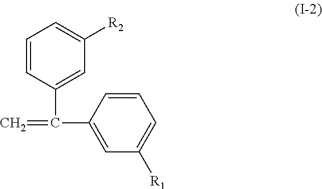

In more specific embodiments of the compounds of structure (I), (I-1) or (I-2), R₁ is said chelating group is a phosphinothioic moiety of structure (Ia). In more specific embodiments of the compounds of structure (I), (I-1) or (I-2), R₁ is said chelating group is an aminosulfonyl moiety of structure (Ib). In more specific embodiments of the compounds of structure (I), (I-1) or (I-2), R₁ is said is a phosphonamide moiety of structure (Ic). In one aspect of these embodiments, R₂ is H. In another aspect of these embodiments, R₂ is an aryl. In another aspect of these embodiments, R₂ is an alkylenearyl. In another aspect of these embodiments. R₂ is a C-2 to C-8 alkyleneoxyalkyl. In another aspect of these embodiments, R₂ is a C-2 to C-8 haloalkyl. In another aspect of these embodiments, R₂ is a C-1 to C-8 linear alkyl. In another aspect of these embodiments, R₂ is a C-3 to C-8 branched alkyl. In another aspect of these embodiments, R₂ is a C-3 to C-8 cyclic alkyl. In another aspect of these embodiments, R₂ is a C-1 to C-8 linear alkyloxy. In another aspect of these embodiments, R₂ is a C-3 to C-8 branched alkyloxy. In another aspect of these embodiments, R₂ is a C-3 to C-8 cyclic alkyloxy. In another aspect of these embodiments, R₂ is a phosphinothioic moiety of structure (Ia). In another aspect of these embodiments, R₂ is an aminosulfonyl moiety of structure (Ib). In another aspect of these embodiments, R₂ is a phosphonamide moiety of structure (Ic).

One embodiment of these novel compounds are those having structure (II), wherein R₁₁ is a phosphinothioic moiety of structure (IIa), wherein * designates the attachment point of this moieties to said compound of structure (II), R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

Further, in these compounds of structure (II), L₁ is a linking moiety selected from the group consisting of a direct valence bond, a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein ** designates the attachment points of the L₁ organic linking moiety to the phosphorous in structure (IIa), and * designates where L₁ within the moiety R₁₁ is attached to the carbonyloxy of compound (II).

Further, in these compounds of structure (II), R₁₂ is H or a C-1 to C-4 alkyl.

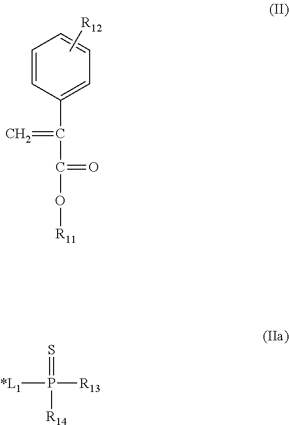

In a more specific embodiments of the aforementioned compounds of structure (II), R₁₃ is a C-1 to C-8 linear alkyloxy. In another aspect of these embodiments, R₁₃ is a C-3 to C-8 branched alkyloxy. In another aspect of these embodiments, R₁₃ is a-C-3 to C-8 cyclic alkyloxy.

In a more specific embodiments of the aforementioned compounds of structure (II), R₁₄ is an aryl. In another aspect of these embodiments, R₁₄ is an alkylenearyl. In another aspect of these embodiments, R₁₄ is a C-2 to C-8 alkyleneoxyalkyl. In another aspect of these embodiments, R₁₄ is a C-2 to C-8 haloalkyl. In another aspect of these embodiments, R₁₄ is a C-1 to C-8 linear alkyl. In another aspect of these embodiments, R₁₄ is a C-3 to C-8 branched alkyl. In another aspect of these embodiments, R₁₄ is a C-3 to C-8 cyclic alkyl. In another aspect of these embodiments, R₁₄ is a C-1 to C-8 linear alkyloxy. In another aspect of these embodiments, R₁₄ is a C-3 to C-8 branched alkyloxy. In another aspect of these embodiments, R₁₄ is, a C-3 to C-8 cyclic alkyloxy. In another aspect of these embodiments, R₁₄ is a dialkyl amino moiety, —N(R₉)(R₁₀).

In another more specific embodiment R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl.

In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl. In another aspect of this embodiment R₁₃ and R₁₄ are selected from a C-1 to C4 alkoxy. In another aspect of this embodiment R₁₃ is selected from a C-1 to C4 alkoxy and R₁₄ is selected from a C-1 to C4 alkyl.

In more specific embodiments of the aforementioned compounds of structure (II), R₉ is a C-1 to C-8 linear alkyl. In another aspect of these embodiments, R₉ is a C-3 to C-8 branched alkyl.

In another aspect of these embodiments, R₉ is a C-3 to C-8 cyclic alkyl.

In more specific embodiments of the aforementioned compounds of structure (II), L₁ is direct valence bond. In another aspect of these embodiments, L₁ is a C-2 to C-8 alkylene moiety. In another aspect of these embodiments, L₁ is an arylene moiety (-aryl-). In more specific embodiments of these embodiments, L₁ is an alkyleneoxyaryl moiety (*-alkylene-O-aryl-**).

In more specific embodiments of the aforementioned compounds of structure (II), it has structure (IIc) or structure (IIc1), where n is 1 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C4 alkyl or alkoxy. In another aspect of this embodiment R₁₃ and R₁₄ are selected from a C-1 to C4 alkyl. In another aspect of this embodiment R₁₃ and R₁₄ are selected from a C-1 to C4 alkoxy. In another aspect of this embodiment R₁₃ is selected from a C-1 to C4 alkoxy and R₁₄ is selected from a C-1 to C4 alkyl.

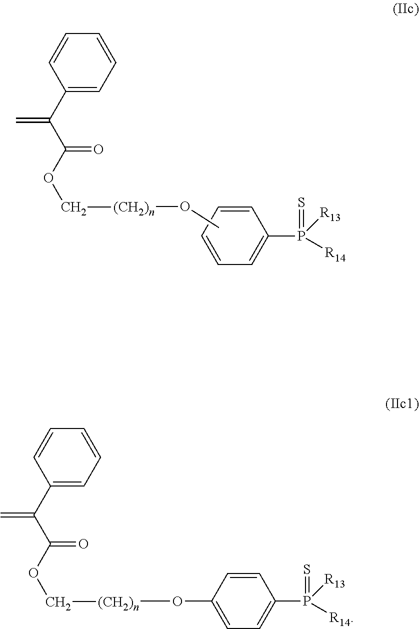

In more specific embodiments of the aforementioned compounds of structure (II), it has structure (IIb) or structure (IIb1), wherein n is an integer ranging from 1 to 7. In one aspect of these embodiments, R₁₃ and R₁₄ are independently selected from a C-1 to C-8 alkyl or alkoxyl. In another aspect of this embodiment R₁₃ and R₁₄ are independently selected from a C-1 to C-4 alkyl or alkoxy. In another aspect of this embodiment R₁₃ and R₁₄ are selected from a C-1 to C-4 alkyl. In another aspect of this embodiment R₁₃ and R₁₄ are selected from a C-1 to C-4 alkoxy. In another aspect of this embodiment R₁₃ is selected from a C-1 to C-4 alkoxy and R₁₄ is selected from a C-1 to C-4 alkyl.

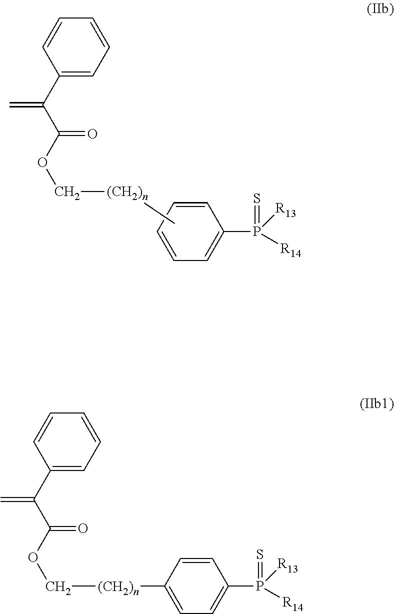

In more specific embodiments of the aforementioned compounds of structure (II), it has structure (IIb2), wherein R₁₃ₐ and R₁₄ₐ and individually selected from a C-1 to C4 alkyl.

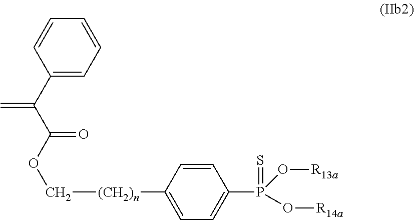

### EXAMPLES

Reference will now be made to more specific embodiments of the present disclosure and experimental results that provide support for such embodiments. The examples are given below to more fully illustrate the disclosed subject matter and should not be construed as limiting the disclosed subject matter in any way.

It will be apparent to those skilled in the art that various modifications and variations can be made in the disclosed subject matter and specific examples provided herein without departing from the spirit or scope of the disclosed subject matter. Thus, it is intended that the disclosed subject matter, including the descriptions provided by the following examples, covers the modifications and variations of the disclosed subject matter that come within the scope of any claims and their equivalents.

Although the disclosed and claimed subject matter has been described and illustrated with a certain degree of particularity, it is understood that the disclosure has been made only by way of example, and that numerous changes in the conditions and order of steps can be resorted to by those skilled in the art without departing from the spirit and scope of the disclosed and claimed subject matter.

### Chemicals

All chemicals unless otherwise indicated were purchased from Sigma Aldrich (3050 Spruce St., St. Louis, MO 63103). Chemicals used in anionic polymerization were purified as described in the literature (e.g. Techniques in High-Vacuum Anionic Polymerization” By David Uhrig and Jimmy Mays and Journal of Polymer Science: Part A: Polymer Chemistry, Vol. 43, 6179-6222 (2005))

Phenyl acrylate derivatives were synthesized by esterification of acryloyl chloride with corresponding hydroxyl compound under basic condition and DPE derivatives were synthesized by alkoxylation of DPE-(m)-CH₂Br (1-(bromomethyl)-3-(1-phenylvinyl)benzene) with corresponding hydroxyl compound under basic condition.

All synthetic experiments were carried out under N₂ atmosphere. Lithographic experiments were carried out as described in the text. The molecular weight of the copolymers was measured with a Gel Permeation Chromatograph. Gel permeation chromatography equipped with 100 Å, 500 Å, 10³ Å, 10⁵ Å and 10⁶ Å μ-ultrastyragel columns

Lithographic Experiments were done using a TEL Clean ACT8 track. SEM pictures were taken with an applied Materials NanoSEM_3D Scanning electron microscope picture are shown at either 1 FOV magnification or 2 FOV magnification (Field of view (FOV)=5 μm).

Etching experiments were done using standard isotropic oxygen etching conditions for self-assembled films block copolymer of methyl methacrylate and styrene.

Gel permeation chromatography equipped with styrene-DVB gel, 100 Å, 500 Å, 103 Å, 104 Å and 106 Å porosities columns connected in series (PSS (polymer standard services), Germany) and THF as mobile phase at 1 ml/min. Polystyrene standards (18 samples) ranging Mₙ from 1,000 g/mol to 200,000 g/mol (PSS, Germany) were used to construct a calibration curve for determining molecular weight and poly distribution index (PDI) of the synthesized polymers. Bruker 400 M Hz NMR unit was used for molecular characterization.

¹H NMR spectra were recorded using Bruker Advanced III 400 MHz spectrometer in CDCl₃.

Coating studies were performed using blanket dielectric as well as metal coupons. The films were baked at desired temperature and time and rinsed with excess material. The grafted brushes were studied using water contact-angle, FT, and XPS.

Coating of polymeric brushes on a blanket metal or dielectric wafers using 230° C./5 min baking condition and rinsing excess material out with organic solvents were done and the substrates were analyzed using WCA, FT and XPS.

### Experimental

### Preparation of Polymer Formulation

The polymers described here were separately dissolved in PGMEA to form 1 wt. % solutions. These solutions where individually filtered in using in using a Nylon filter (Entegris, Billerica, Ma). These solutions were separately coated at 1500 rpm on both metal (Cu, W) and SiO₂ wafers, and the wafers were subsequently baked at 230° C. for 5 min. Following the bake, the wafers were rinsed with PGMEA for 2 min to remove any un-grafted polymer from the wafer which were then spun dried by spinning “1,500 rpm,” followed by baking at 110° C. for 1 min. Then water contact angle, XPS were measured to understand the grafting efficiency and the results were shown in Table 1. Subsequently, the second brush of hydroxyl terminated PS—OH or PMMA-OH containing polymer formulation was made in PGMEA at 1 wt. % solid. Then after filtering with 0.25-micron Nylon filter, the solution was spin coated on to previously brushed metal and SiO₂ substrates. After baking at various temp. and time, the double brushed substrates were rinsed to remove unreacted second brushes. Then the double brushed substrates were examined by water contact angle (WCA) and X-ray photoelectron spectroscopy (XPS) to understand cross-grafting to judge the first brush's efficiency and selectivity to metal substrates.

### Example 1 Synthesis of Dimethyl Thiophophine Terminated Polystyrene

Anionic polymerization was performed in nitrogen dried 250 mL round bottom flask equipped with, a magnetic stir bar, a septum adaptor that enables connection to either vacuum or nitrogen atmosphere, and a rubber septum for reagent addition via using syringe or cannula. Anhydrous cyclohexane, 100 mL was transferred into the flask using a cannula under nitrogen. Purified styrene monomer, 20 mL (18 g) was added to the cyclohexane solution. The reaction mixture was degassed and filled with nitrogen.

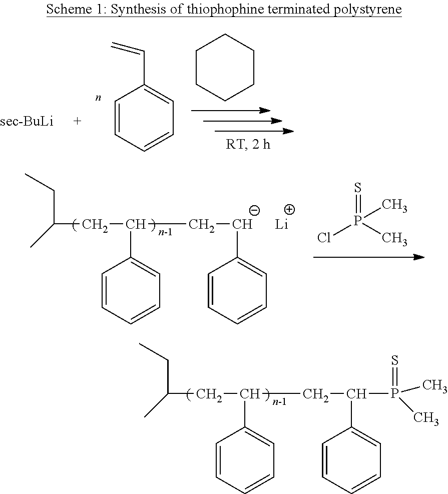

A required amount of initiator, sec-butyl lithium (1.3M) solution in hexane was added into the monomer solution under fast stirring. Immediately, an orange color of carbanion formed indicating propagation of polystyryl lithium with warmness of the reaction increased slightly. Reaction was kept stirring at 35° C.-45° C. for 2 h. Then, a purified dimethyl thiophosphinoyl chloride (distilled over CaH₂) was transferred via cannula into the living polystyryl lithium anion solution. The color of the reaction slowly disappeared over 5 mins. The reaction mixture was diluted with THF (2×) and then precipitated into excess (5×) isopropanol:water (80:20) mixture. The polymer was filtered and dried under vacuum at 80° C., over 18 h. Yield: 17.2 grams (96%), Mₙ, GPC=6,600 g/mol, PDI=1.05, ³¹P NMR showed a signal at 44 ppm confirming presence of end group with —P═S. A solution of this polymer in PGMEA (1 wt. %) spin-coated on to the tungsten and silicon coupons after bake (200° C./15 min/N₂) and rinse (2 min, dynamic) showed 90° on W and 52° on Silicon coupons, respectively. This confirms the selectivity of this polymer to W. XPS studies showed elemental ratio of C/W and C/Si as 25.84 and 0.43, respectively.

### Example 2. Synthesis of Diethylthiophosphonate Terminated Polymethyl Methacrylate

In the first step, the diethylthiophosphonate DPE derivative was prepared from the thiolation of MTAG-10. Diethylphosphonate terminated PMMA was synthesized using an initiator made from diethylthiophosphonate DPE derivative and sec-BuLi. The reaction was carried out at −78° C. in tetrahydrofuran.

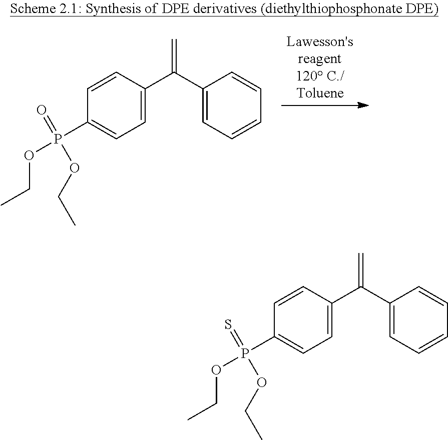 [SMILES: C=C(c1ccccc1)c1ccc(P(=O)(OCC)OCC)cc1 ; C=C(c1ccccc1)c1ccc(P(=S)(OCC)OCC)cc1]

### Step 1: Synthesis of Diethylthiophosphonate DPE

Diethylphosphonate DPE (MTAG-10) (29.0 g, 91.67 mmol) and Lawesson's reagent (18.7 g, 404.471 mmol) were weighed in two necks round bottom flask, attached with a reflux condenser and rubber septum. 250 mL anhydrous toluene was cannula transferred. Flask was kept in an oil bath and the temperature increased to 120° C. The reaction was left to run overnight (although, 2-4 hr are enough). Toluene was removed on rotovap. The brownish-red liquid was loaded on a silica column and separated using a mixture of hexane:ethyl acetate with a 70:30 ratio. Excess Lawesson reagent and its side products elute very close to the desired product. Desired DPE derivative (O,O-diethyl (4-(1-phenylvinyl)phenyl)phosphonothioate [DPE-PS(OEt)₂] was obtained as a pale-yellowish-colored liquid. Lawesson reagent and MTAG-26 have a very stinky odor, a typical of thiol compounds. Yield: 20g, % yield 65.6%. ¹H NMR, CDCl₃ δ: 1.4, dt, 6H; 4.0, m, 4H; 5H; 7.3m, 2H; 7.4, 2H, 7.6, m, 2H., 31P CPD m, 87.4 ppm

### Step 2: Synthesis of PMMA Terminated with Diethylthiophosphonate

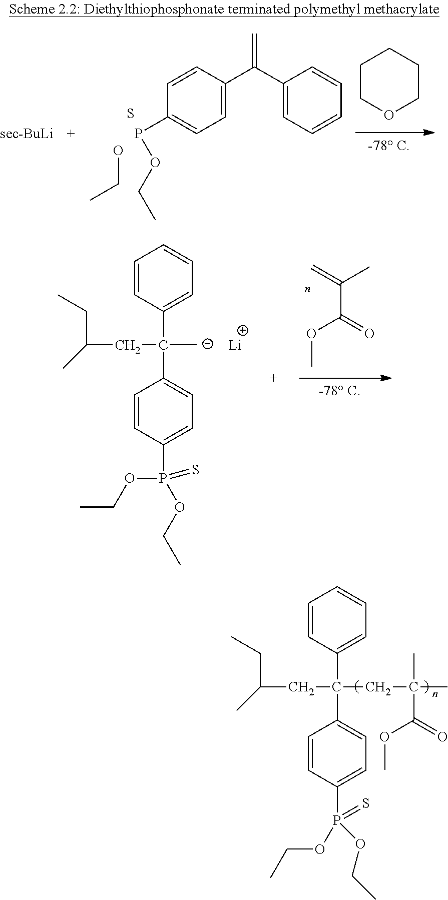

O,O-diethyl (4-(1-phenylvinyl)phenyl)phosphonothioate [DPE-PS(OEt)₂](1.72 g, 5 mmol) was dissolved in hexyl lithium titrated 6 ml toluene and added into an ampule. 28 mL methyl methacrylate was added into a separate ampule and degassed under a dynamic vacuum to 25 mL (23.3 g, 23.3 mmol) of methyl methacrylate. The ampules were attached to the sidearms of the reactor containing lithium chloride (2.2 g, 41 mmol) and a magnetic stir bar. The reactor was evacuated under a vacuum and charged with nitrogen. Tetrahydrofuran (300 mL) was added via cannula and the mixture was stirred and cooled to −78° C. where the mixture was titrated with sBuLi until a yellow color persists. The mixture was warmed to room temperature where the yellow color dissipates. The mixture was cooled to −78° C. and the DPE-P(S)(OEt)₂) solution was added. sBuLi (4.0 mL, 1.17M in cyclohexane, 4.68 mmol) was then added slowly to produce a red mixture. Methyl methacrylate was added dropwise over 6 minutes at which the red color turns colorless. The mixture was stirred for an additional 12 minutes at which time, 1 mL of degassed methanol was added to terminate the reaction. The polymer brush was recovered by precipitation in excess hexane (7 times of the polymer solution), filtered, and dried at 40° C. for 1 h. Redissolved in ethyl acetate and washed with Milli-Q water. Ethyl acetate was rotovated and the polymer was further dried under vacuum to give a white powder (22 g, 97% yield). GPC: 4,600 g/mol Mₙ, 4,460 g/mole Mw, 1.046 PDI., ¹H NMR MW 6,000 g/mol. The presence of the end-functional group was confirmed using ¹HNMR and ³¹P NMR analysis.

### Example 3 Synthesis of Diethylthiophosphonate Terminated Polystyrene

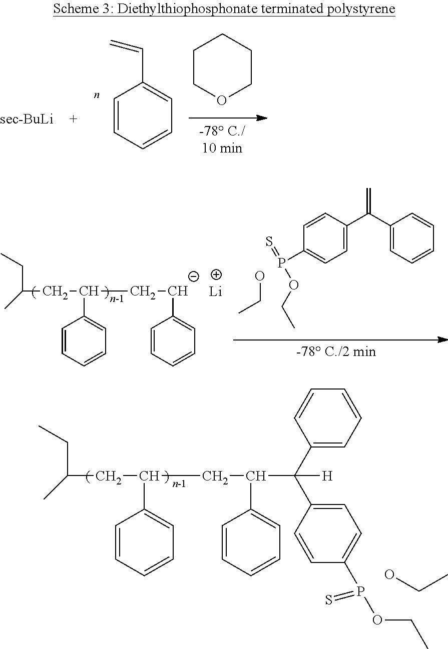

(2.0 g, 3.322 mmol) diethylthiophosphonate DPE was dissolved in 6 ml toluene, titrated with 1,1-diphenyl-3-methylpentyllithium (made from the reaction of sec-butyllithium with DPE) and added into an ampule. 28 mL styrene was added into a separate ampule and degassed under a dynamic vacuum to 25 mL (24.543 g, 23.65 mmol) of styrene. The ampules were attached to the sidearms of the reactor containing lithium chloride (2.2 g, 41 mmol) and a magnetic stir bar. The reactor was evacuated under a vacuum and charged with nitrogen. Tetrahydrofuran (300 mL) was added via cannula and the mixture was stirred and cooled to −78° C. where the mixture was titrated with sBuLi until a yellow color persists. The mixture was warmed to room temperature where the yellow color dissipates. The mixture was cooled to −78° C. and sBuLi (4.0 mL, 1.17M in cyclohexane, 4.68 mmol) was added to produce a pale-yellow mixture. Styrene was added dropwise over 10 minutes at which the orange color forms. The mixture was stirred for an additional 5 minutes at which time, the DPE-P(S)(OEt)₂) solution was added. Red colored solution was stirred for few minutes and reaction was terminated with 1 mL degassed methanol.

The polymer recovered by precipitation in excess isopropanol (7 times of the polymer solution), filtered, and dried at 40° C. for 1 h. Redissolved in ethyl acetate and washed with Milli-Q water and precipitate in excess isopropanol. Filtered and dried under vacuum to give a white powder (24 g, 92% yield). GPC: 3,900 g/mol Mₙ, 4,800g/mole Mw, 1.14 PDI., ¹H NMR MW 4,700 g/mol. Presence of end-functional group was confirmed using ¹HNMR and ³¹P NMR analysis.

### Example 4 Synthesis of Ethyl Dimethylphosphonamide Terminated Polymethyl Methacrylate (

### 4.1 Synthesis of 1-phenyl-1′-[4-ethyl(dimethylphosphonamido)]-phenylethylene

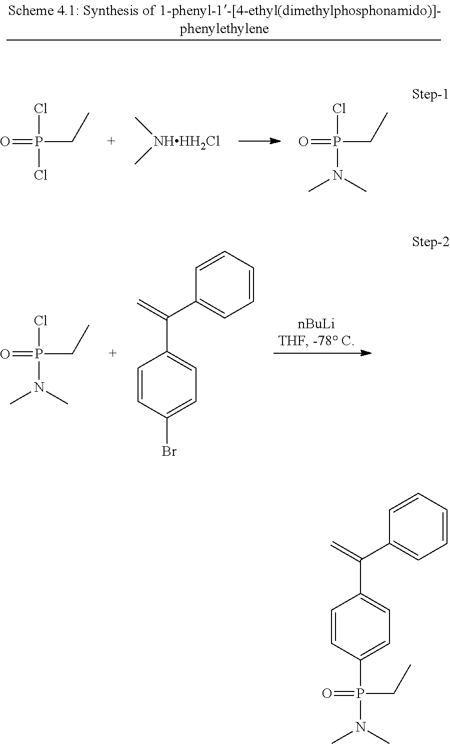

4-Bromobenzophenone (25 g, 95.7 mmol) and THF were stirred together and cooled to 0° C. nButyllithium (71.38 mL, 114.9 mmol) was added, followed by methyl triphenylphosphonium bromide (41 g, 114.9 mmol). The mixture was warmed to RT. The reaction was quenched with water and diluted with ethyl acetate. The mixture was washed with 1% aq. HCl and aq. NaCl solution and dried over MgSO₄. The MgSO₄ was filtered out and the filtrate was concentrated in vacuo. The white solid triphenylphosphonium oxide was precipitated by slurring in hexane: ethyl acetate (2:1) and then filtered off. Then the filtrate was concentrated and purified by silica gel column chromatography using hexane as eluant followed by concentration to give 4-bromo-1,1′-diphenylethylene as a colorless oil (18g % yield 72%)

Step-1: Ethylphosphonic dichloride (1.70 mL, 15.9 mmol) and dimethylammonium chloride (1.30 g, 16.0 mmol) were dissolved in dichloromethane and cooled to 0° C. in an ice-water bath. Triethylamine (4.46 mL, 32.0 mmol) was added, and the mixture warmed to RT for 30 minutes. The slurry was filtered, and the filtrate concentrated in-vacuo. The residue was redissolved in dichloromethane and triturated with ether. The mixture was filtered and concentrated, then repeated to remove the salt. This gave the chloroethyl(dimethylphosphonamido) as a sticky solid (2.3 g, 95% yield).

Step-2: 4-Bromo-1,1′-diphenylethylene generated from (a) (1 g, 3.86 mmol) was dissolved in tetrahydrofuran and cooled to −78° C. nBuLi (2.4 mL, 3.86 mmol, 1.6M in hexane) was added and stirred 30 minutes. In a separate flask, the chloroethyl(dimethylphosphoramide) generated from (b) (0.6 g, 3.86 mmol) was slurred in THF and cooled to −78° C. The 4-lithium-1,1′-diphenylethylene generated previously was added in by cannula and the mixture warmed to RT and stirred for 72 hours. The mixture was diluted with ethyl acetate, washed with water, and dried over MgSO₄. The MgSO₄ was filtered out and the filtrate concentrated in vacuo and purified by silica gel column chromatography using 50% ethyl acetate in hexane as eluant. Concentration of the product fractions gave 1-phenyl-1′-[4-ethyl(dimethylphosphonamido)]-phenylethylene as a pale-yellow oil (0.7g yield 60%)¹H NMR, CDCl₃ δ: 1.10, dt, 3H; 1.98, m, 2H; 2.66, s, 3H; 2.69, s, 3H; 5.53, s, 2H; 7.33, m, 5H; 7.40, m, 2H; 7.68, m, 2H.

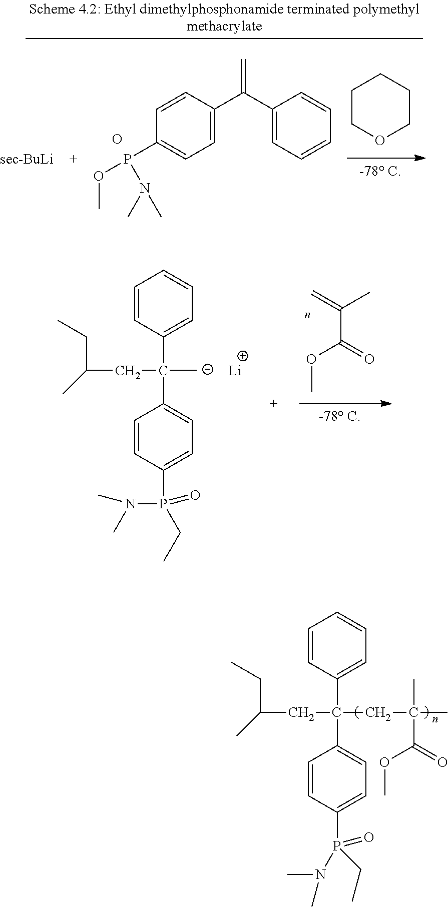

### 4.2 Synthesis of PMMA-ethyl(dimethylphosphonamide) Terminated

1-Phenyl-1′-(4-ethyldimethylphosphonamido)phenylethylene [DPE-P(O)(Et)(NMe₂)] (1.43 g, 5 mmol) in 6 ml of dry toluene was added into an ampule. Methyl methacrylate (20.7 g, 207 mmol) was added into a separate ampule and freeze-thawed 3 times to degas. The ampules were attached to the sidearms of the reactor containing lithium chloride (1.75 g, 41 mmol) and a magnetic stir bar. The reactor was evacuated under vacuum and charged with nitrogen. Tetrahydrofuran (210 mL) was added via cannula and the mixture stirred and cooled to −78° C. where the mixture was titrated with sBuLi until a yellow color persists. The mixture was warmed to room temperature where the yellow color dissipates. The mixture was cooled to −78° C. once again and the DPE-P(O)(Et)(NMe₂) solution was added in. sBuLi (1.2 mL) was added dropwise to titrate the mixture until a yellow color persists. sBuLi (2.96 mL, 1.4M in cyclohexane, 4 mmol) was then added slowly to produce a red mixture. Methyl methacrylate was added rapid dropwise to the mixture over 2 minutes at which the red color turns colorless. The mixture was stirred for an additional 30 minutes at which time, 1 mL of degassed methanol was added to terminate the reaction. The polymer brush was recovered by precipitation in excess hexane (7 times of the polymer solution), filtered, and dried at 40° C. for 12 h under vacuum to give a white powder (21 g, 97% yield). GPC: 4,195 g/mol Mₙ, 4,460 g/mole Mw, 1.06 PDI.

### Example 5. Synthesis of Phenyl Dimethyl Phosphonamide Terminated Polymethyl Methacrylate

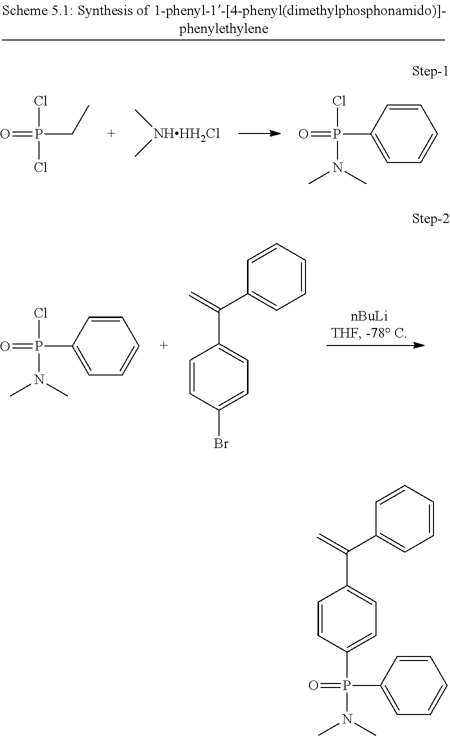

### 5.1 Synthesis of 1-phenyl-1′-[4-phenyl(dimethylphosphonamido)]-phenylethylene

Step-1: 4-Bromobenzophenone (25 g, 95.7 mmol) and THF were stirred together and cooled to 0° C. nButyllithium (71.38 mL, 114.9 mmol) was added, followed by methyl triphenylphosphonium bromide (41 g, 114.9 mmol). The mixture was warmed to RT. The reaction was quenched with water and diluted with ethyl acetate. The mixture was washed with 1% aq. HCl and aq. NaCl solution and dried over MgSO₄. The MgSO₄ was filtered out and the filtrate was concentrated in vacuo. The white solid triphenylphosphonium oxide was precipitated by slurring in hexane: ethyl acetate (2:1) and then filtered off. Then the filtrate was concentrated and purified by silica gel column chromatography using hexane as eluant followed by concentration to give 4-bromo-1,1′-diphenylethylene as a colorless oil 20g, 80% ield)

Step-2: Phenylphosphonic dichloride (15 mL, 105.8 mmol) and dimethylammonium chloride (8.64 g, 106.0 mmol) were dissolved in dichloromethane and cooled to 0° C. in an ice-water bath. Triethylamine (29.5 mL, 211.6 mmol) was added, and the mixture was warmed to RT for 30 minutes. The slurry was filtered, and the filtrate concentrated in-vacuo. The residue was redissolved in dichloromethane and triturated with ether. The mixture was filtered and concentrated, then repeated to remove the salt. This gave the chlorophenyl(dimethylphosphoramide) a sticky solid (12 g, 80% yield)

Step-3: 4-Bromo-1,1′-diphenylethylene generated from 5.1 (3 g, 11.6 mmol) was dissolved in tetrahydrofuran and cooled to −78° C. nBuLi (8.7 mL, 13.9 mmol, 1.6M in hexane) was added and stirred 30 minutes. In a separate flask, the chlorophenyl(dimethylphosphoramide) generated from (b) (3.4 g, 16.7 mmol) was slurried in THF and cooled to −78° C. The 4-lithium-1,1′-diphenylethylene generated previously was added in by cannula and the mixture warmed to RT and stirred for 72 hours. The mixture was diluted with ethyl acetate, washed with water, and dried over MgSO₄. The MgSO₄ was filtered out and the filtrate concentrated in vacuo and purified by silica gel column chromatography using 50% ethyl acetate in hexane as eluant. Concentration of the product fractions gave 1-phenyl-1′-[4-phenyl(dimethylphosphonamido)]-phenylethylene as a pale-yellow oil (3.5 g, 70% yield)¹H NMR, CDCl₃ δ: 2.68, s, 3H; 2.70, s, 3H; 5.50, s, 2H; 7.30, m, 5H; 7.47, m, 5H; 7.80, m, 4H.

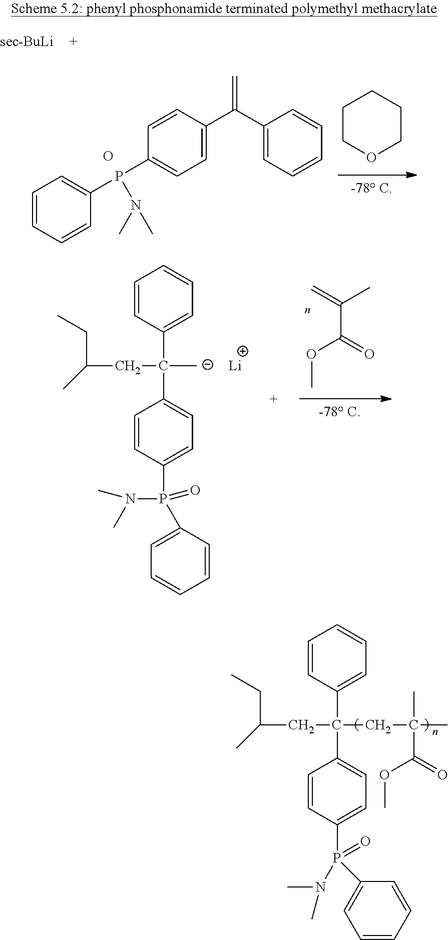

### 5.2: Synthesis of Phenyl Dimethyl Phosphonamide Terminated Polymethyl Methacrylate

1-Phenyl-1′-(4-phenyldimethylphosphonamido)phenylethylene[DPE-P(O)(Ph)(NMe₂)] (1.58 g, 5 mmol) of in 6 ml of dry toluene was added into an ampule. Methyl methacrylate (20.68 g, 207 mmol) was added into a separate ampule and freeze-thawed 3 times to degas. The ampules were attached to the sidearms of the reactor containing lithium chloride (1.75 g, 41 mmol) and a magnetic stir bar. The reactor was evacuated under vacuum and charged with nitrogen. Tetrahydrofuran (210 mL) was added via cannula and the mixture stirred and cooled to −78° C. where the mixture was titrated with sBuLi until a yellow color persists. The mixture was warmed to room temperature where the yellow color dissipates. The mixture was cooled to −78° C. once again and the DPE-P(O)(Ph)(NMe₂) solution was added in. sBuLi (1.2 mL) was added dropwise to titrate the mixture until a yellow color persists. sBuLi (3 mL, 1.4M in cyclohexane, 4 mmol) was then added slowly to produce a red mixture. Methyl methacrylate was added rapid dropwise to the mixture over 2 minutes at which the red color turns colorless. The mixture was stirred for an additional 30 minutes at which time, 1 mL of degassed methanol was added to terminate the reaction. The polymer brush was recovered by precipitation in excess hexane (7 times of the polymer solution), filtered, and dried at 40° C. for 12 h under vacuum to give a white powder (21 g, 99% yield). GPC: 5,602 g/mol Mₙ, 6,158 g/mole Mw, 1.10 PDI.

### Example 6. Synthesis of Dimethylsulfonamide Terminated Polymethyl Methacrylate

### 6.1 Synthesis of 1-phenyl-1′-[4-(dimethylsulfonamido)]-phenylethylene

Step-1: Acetophenone (3.31 mL, 28.4 mmol) was dissolved in tetrahydrofuran and cooled to −78° C. nBuLi (12.6 mL, 20.2 mmol, 1.6M in hexane) was added, stirred for 30 minutes, 4-bromobenzenedimethylsulfonamide (4.85 g, 18.4 mmol) was added and the mixture was stirred for 30 minutes, then warmed to RT for 1 hour. The mixture was diluted with ethyl acetate, washed with water, and dried over MgSO₄. The MgSO₄ was filtered out and the filtrate was concentrated in vacuo and purified by silica gel column chromatography using 33% ethyl acetate in hexane as eluant. The concentration of the product fractions gave 1-phenyl-1-[4-(dimethylsulfonamido)]-phenylethan-1-ol as a white solid (2.7 g, 48% yield).

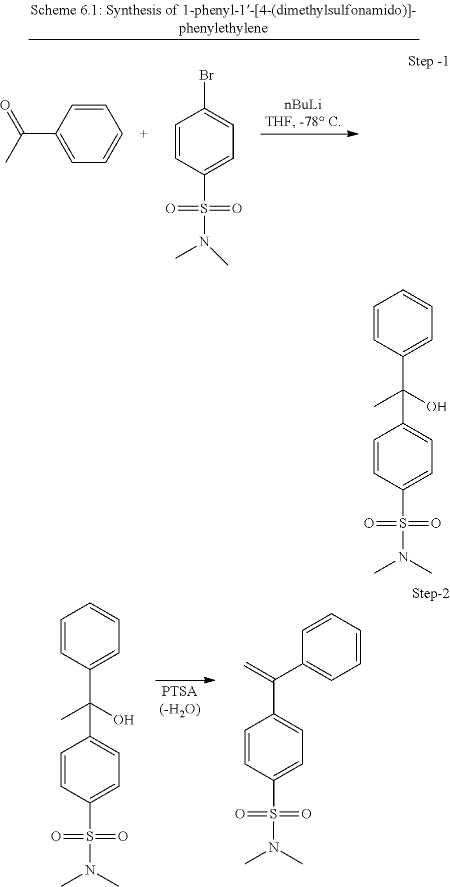

Step-2: 1-Phenyl-1-[4-(dimethylsulfonamido)]-phenylethan-1-ol generated from (a) (2.7 g, x mmol) and p-toluenesulfonic acid monohydrate (cat.) were slurred in toluene and heated to 70° C. for 3 hours. The mixture was diluted with ethyl acetate and washed with water and dried over MgSO₄. The MgSO₄ was filtered out and the filtrate was concentrated in vacuo and purified by silica gel column chromatography using 33% ethyl acetate in hexane as eluant. Concentration of the product fractions gave 1-phenyl-1′-[4-(dimethylsulfonamido)]-phenylethylene as a pale yellow oil (2.6 g, 99% yield). ¹H NMR, CDCl₃ δ: 2.74, s, 6H; 5.56, s, 1H; 5.59, s, 1H; 7.30, m, 2H; 7.36, m, 3H; 7.50, d, 2H; 7.74, d, 2H 6:2: PMMA-brush synthesis:

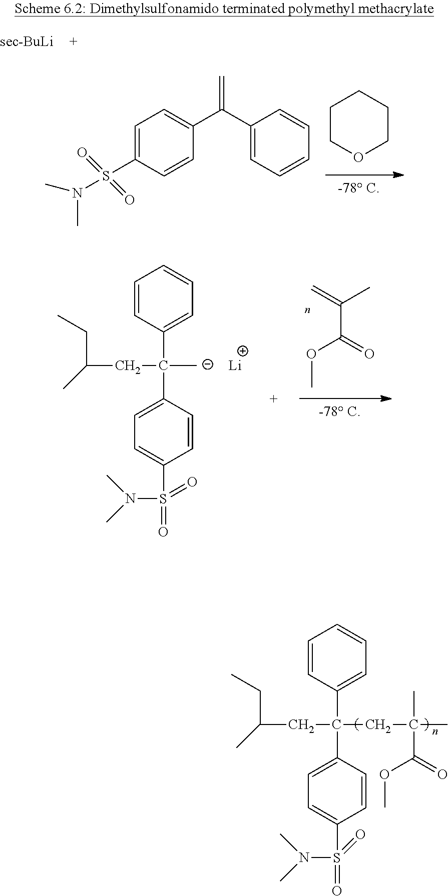

1-Phenyl-1′-[4-(dimethylsulfonamido)]-phenylethylene [DPE-SO₂(NMe₂)](1.02 g, 3.6 mmol) in 3 ml of dry toluene was added into an ampule. Methyl methacrylate (15.8 g, 157 mmol) was added into a separate ampule and freeze-thawed 3 times to degas. The ampules were attached to the sidearms of the reactor containing lithium chloride (0.4 g, 10 mmol) and a magnetic stir bar. The reactor was evacuated under vacuum and charged with nitrogen. Tetrahydrofuran (200 mL) was added via cannula and the mixture stirred and cooled to −78° C. where the mixture was titrated with sBuLi until a yellow color persists. The mixture was warmed to room temperature where the yellow color dissipates. The mixture was cooled to −78° C. once again and the DPE-SO₂(NMe₂) solution was added in. sBuLi (1.2 mL) was added dropwise to titrate the mixture until a yellow color persists. sBuLi (2.1 mL, 1.4M in cyclohexane, 3 mmol) was then added slowly to produce a red mixture. Methyl methacrylate was added rapid dropwise to the mixture over 2 minutes at which the red color turns colorless. The mixture was stirred for an additional 30 minutes at which time, 1 mL of degassed methanol was added to terminate the reaction. The polymer brush was recovered by precipitation in excess hexane (7 times of the polymer solution), filtered, and dried at 40° C. for 12 h under vacuum to give a white powder (15 g, 95% yield). GPC: 5,773 g/mol Mₙ, 5,946 g/mole Mw, 1.03 PDI.

### Preparation of Polymer Formulation

The polymers described here were separately dissolved in PGMEA to form 1 wt. % solutions. These solutions where individually filtered in using in using a Nylon filter (Entegris, Billerica, Ma). These solutions were separately coated at 1500 rpm on both metal (Cu, W) and SiO₂ wafers, and the wafers were subsequently baked at 230° C. for 5 min. Following the bake, the wafers were rinsed with PGMEA for 2 min to remove any un-grafted polymer from the wafer which were then spun dried by spinning “1,500 rpm,” followed by baking at 110° C. for 1 min. Then water contact angle, XPS were measured to understand the grafting efficiency and the results were shown in Table 1. Subsequently, the second brush of hydroxyl terminated PS—OH or PMMA-OH containing polymer formulation was made in PGMEA at 1 wt. % solid. Then after filtering with a 0.25-micron Nylon filter, the solution was spin coated on to previously brushed metal and SiO2 substrates. After baking at various temp. and time, the double brushed substrates were rinsed to remove unreacted second brushes. Then the double brushed substrates were examined by WCA and XPS to understand cross-grafting to judge the first brush's efficiency and selectivity to metal substrates.

### Coating of Brushes:

Coating of polymeric brushes on a blanket metal or dielectric wafers using 230° C./5 min baking condition and rinsing excess material out with organic solvents were done and the substrates were analyzed using WCA, FT and XPS.

Table 1 shows data for selective grafting of metals with different materials which showed high carbon ratios with substrates and appropriate high contact angles depending on the polarity of grafted polymer on metal. Polymers were coated at 1% solution in PGMEA and baked at 230° C./5 min and then rinsed for 2 mins with PGMEA solvent. All the polymers used in these tests had an Mₙ of around 5,000 g/mol.

TABLE 1

| | | At % C 1 s | | C/W or C/Si | | WCA | |
|---|---|---|---|---|---|---|---|
| Materials | Name/Mn | W | SiOx | W | SiOx | W | SiOx |
| Example 1 | 5k | 84.6 | 22.5 | 25.84 | 0.43 | 90 | 52 |
| PS—P═S(Me)₂ | | | | | | | |
| Example 2 | 4.6K | 43.9 | 5.9 | 14.5 | 0.10 | 67 | 39 |
| PMMA—DPE—P═S(OEt)₂ | | | | | | | |
| Example 3 | 4.8K | 83.0 | 26.3 | 50.6 | 0.56 | 90 | 70 |
| PS—DPE—P═S(OEt)₂ | | | | | | | |
| Example 4 | 4.2k | 63.1 | 25.5 | 7.35 | 1.00 | 66 | 65 |
| PMMA—DPE—P═OEt N(Me)₂ | | | | | | | |
| Example 5 | 4.5k | 48.7 | 3.42 | 2.56 | 0.10 | 87 | 52 |
| PMMA—DPE—P(═O)Ph N(Me)₂ | | | | | | | |
| Example 6 | 5.8k | 31.2 | 2.44 | 1.3 | 0.07 | 62 | 44 |
| PMMA—DPE—S(═O)₂(NMe₂) | | | | | | | |

### Preparation of Polymer Formulation

The polymers described here were separately dissolved in PGMEA to form 1 wt. % solutions. These solutions where individually filtered in using in using a Nylon filter (Entegris, Billerica, Ma). These solutions were separately coated at 1500 rpm on both metal (Cu, W) and SiO₂ wafers, and the wafers were subsequently baked at 230° C. for 5 min. Following the bake, the wafers were rinsed with PGMEA for 2 min to remove any un-grafted polymer from the wafer which were then spun dried by spinning “1,500 rpm,” followed by baking at 110° C. for 1 min. Then water contact angle, XPS were measured to understand the grafting efficiency and the results were shown in Table 1. Subsequently, the second brush of hydroxyl terminated PS—OH or PMMA-OH containing polymer formulation was made in PGMEA at 1 wt. % solid. Then after filtering with 0.25-micron Nylon filter, the solution was spin coated on to previously brushed metal and SiO₂ substrates. After baking at various temp. and time, the double brushed substrates were rinsed to remove unreacted second brushes. Then the double brushed substrates were examined by WCA and XPS to understand cross-grafting to judge the first brush's efficiency and selectivity to metal substrates.

1. A polymer of structure (A), comprising two end groups R₃ₚ and R₄ₚ, and a polymer chain (R) comprising either a repeat unit of structure (Ip) or a repeat unit of structure (lip), wherein
end group R₃ₚ, which is derived from an anionic initiator, is either a C-1 to C-8 alkyl, a moiety of structure (IIIp), or a moiety of structure (IIIp1), but where R₃ₚ can only be selected from a C-1 to C-8 alkyl if the in the polymer chain (R) the repeat unit is (IIp), and further where R₃ₚ can only be selected from a moiety of structure (IIIp) or structure (IIIp1) if in the polymer chain (R) the repeat unit is of structure (Ip),
end group R₄ₚ is either a moiety selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, a moiety of structure (IVp), a moiety of structure (IVp1) or a moiety of structure (IVp2), further end groups R₃ₚ and R₃ₚ cannot respectively, both simultaneously be moieties of structure (IIIp) and (IVp), both simultaneously be moieties of structure (IIIp) and (IVp1), or both simultaneously be moieties of structure (IIIp) and (IVp2), but where said polymer of structure (A) must contain one end group moiety selected from structures (IIIp), (IVp), (IVp1) or (IVp2),
in said repeat unit of structure (Ip) Rₘ₁ is a C-1 to C-8 alkyl, R₁, is a C-1 to C-8 alkyl, and n1 is the number of this repeat units in polymer chain (R);
in said repeat unit of structure (lip) R₂ is H or a C-1 to C-8 alkyl, R₂ₚ is H or a C-1 to C-8 alkyl, n2 is the number of this repeat unit polymer chain (R); and
in structure (IIIp),
R₁ is a chelating group, located at the para or meta position, selected from a phosphinothioic moiety of structure (Ia) an aminosulfonyl moiety of structure (Ib), a phosphonamide moiety of structure (Ic), where *** designates the attachment point of this end group moiety to the polymer of structure (A), wherein
in said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl, and designates the attachment point of this moiety to an end group moiety of structure of (IIIp),
in said aminosulfonyl moiety of structure (Ib), R₅ and R₆, are independently a C-1 to C-8 linear alkyl, a C-3 to C-8 branched alkyl, a C-3 to C-8 cyclic alkyl, and said dialkyl amino moiety, —N(R₉)(R₁₀), and designates the attachment point of this moiety to an end group moiety of structure of (IIIp),
in said phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy, and designates the attachment point of this moiety to an end group moiety of structure of (IIIp),
R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic), and
R₁₅ is a C-1 to C-8 alkyl, and Rₑ₁ and Rₑ₂ are individually selected from H, a C-1 to C-8 alkyl, and a C-1 to C-8 alkoxy,
in structure (IIIp1),
Rₑ₁ and Rₑ₂ are individually selected from H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy, and R₁₅ is a C-1 to C-8 alkyl, and ** designates the attachment point of this end group moiety to the polymer of structure (A),
in structure (IVp),
R₁₂ is H or a C-1 to C-4 alkyl,
R₁₁ is a phosphinothioic moiety of structure (IIa), wherein designates the attachment point of this phosphinothioic moiety to structure (IVp), wherein
R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which Rs and R₁₀ are independently selected from a C-1 to C-8 linear alkyl, a C-3 to C-8 branched alkyl, a C-3 to C-8 cyclic alkyl,
L₁ is a linking moiety selected from the group consisting of a direct valence bond, a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein * designates the attachment points of the L₁ organic linking moiety to the phosphorous in structure (IIa), and * designates where L₁ within the moiety R₁₁ is attached to carbonyloxy of structure (IVp),
R₁₇ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety, and ** designates the attachment point of this end group moiety to the polymer of structure (A),
in structure (IVp1),
L is either a direct valence bond or a linking group selected from a C-1 to C-8 linear alkylene, C-3 to C-8 branched alkylene, and a C-5 to C-8 cyclic alkylene, an alkyleneoxyaryl moiety (*-alkylene-O-aryl-**), and an alkylenearyl moiety (*-alkylene-aryl-**), wherein * designates the attachment points of the L linking moiety to the phosphorous in structure (IVp1), and * designates where L is attached to said polymer of structure (A), and Rs and Rs1 are individually selected from a C-1 to C-8 alkoxy or a C-1 to C-8 alkyl, and where ** designates the attachment point of this end group moiety to the polymer of structure (A),
in structure (IVp2),
R₁ is a chelating group, located at the para or meta position, selected from said phosphinothioic moiety of structure (Ia) said aminosulfonyl moiety of structure (Ib), and said phosphonamide moiety of structure (Ic),
R₂ is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic), R₁₈ is selected from the group consisting of H, a C-1 to C-8 alkyl, C-1 to C-8 alkylcarbonyl (alkyl-C(═O)—), a C-1 to C-8 trialkylsilyl ((alkyl)₃Si—), a C-1 to C-8 dialkysilyl ((alkyl)₂HSi—), a C-1 to C-8 monoalkylsilyl ((alkyl)H₂Si—), silane (H₃Si—), and a benzylic moiety,
and where *** designates the attachment point of this end group moiety to the polymer of structure (A),
Further, said polymer of structure (A) has a Mₙ ranging from about 4000 to about 7000, and has a polydispersity ranging from 1 to about 1.15;

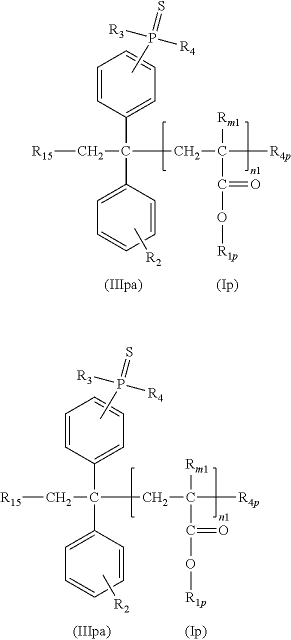

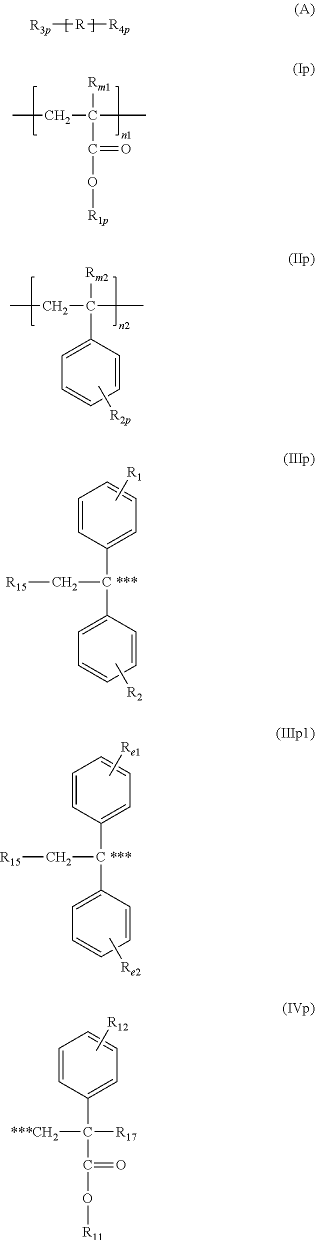

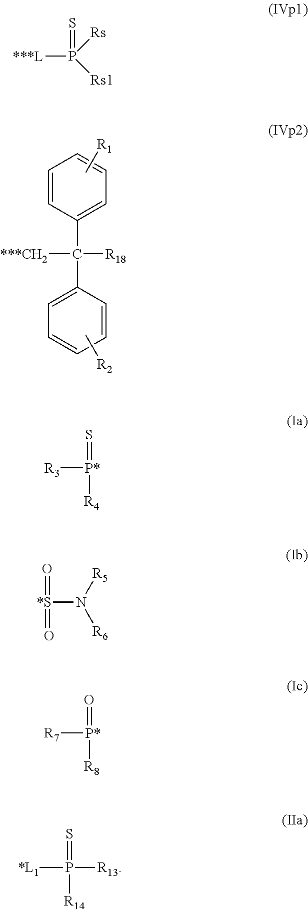

2. The polymer of claim 1, which has structure (A-1),

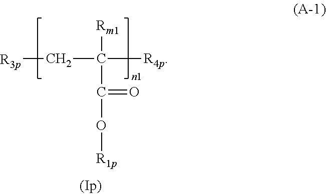

3. The polymer of claim 1, which has structure (A-2),

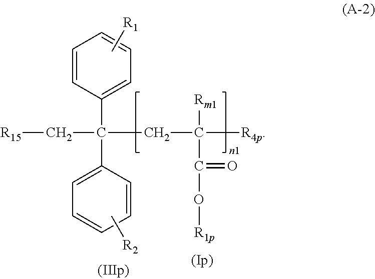

4. The polymer of claim 1, which has structure (A-2a)

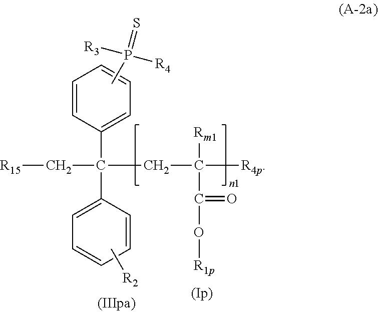

5.-6. (canceled)

7. The polymer of claim 1, which has structure (A-2d),

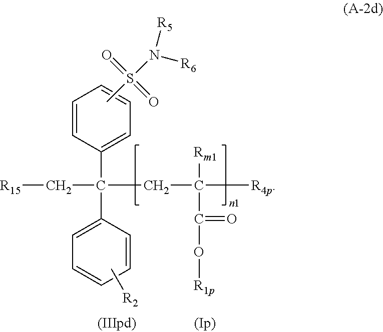

8.-9. (canceled)

10. The polymer of claim 1, which has structure (A-2g),

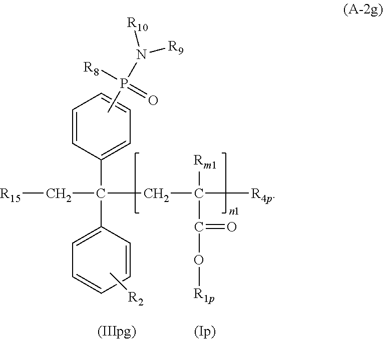

11.-12. (canceled)

13. The polymer of claim 1, which has structure (A-3)

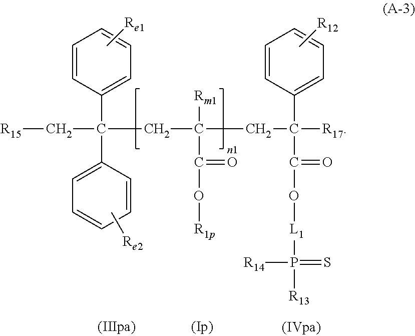

14.-17. (canceled)

18. The polymer of claim 1, which has structure (A-3a) wherein R₁₃ and R₁₄ are independently selected from a C-1 to C-8 alkyl or alkoxy and n is an integer ranging from 1 to 7,

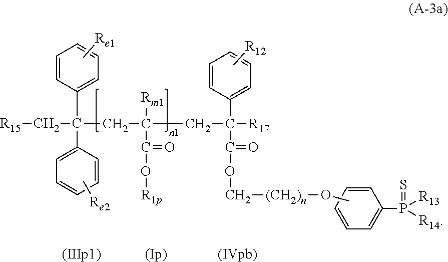

19. (canceled)

20. The polymer of claim 1, which has structure (A-3c), wherein R₁₃ and R₁₄ are independently selected from a C-1 to C-8 alkyl or alkoxy, and n′ is an integer ranging from 0 to 7,

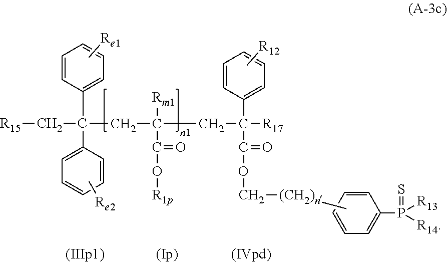

21. (canceled)

22. The polymer of claim 1, which has structure (A-4),

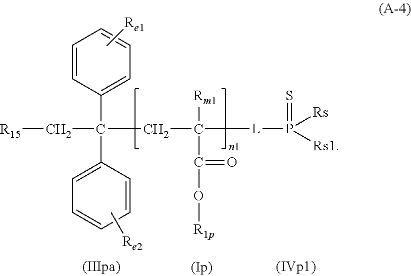

23.-31. (canceled)

32. The polymer of claim 1, which has structure (B-1), wherein R₃ₚ is a C-1 to C-8 alkyl,

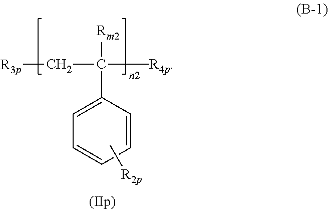

33. The polymer of claim 1, which has structure (B-1a), wherein R₃ₚ is a C-1 to C-8 alkyl,

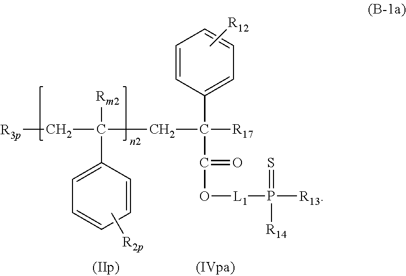

34.-41. (canceled)

42. The polymer of claim 1, which has structure (B-2),

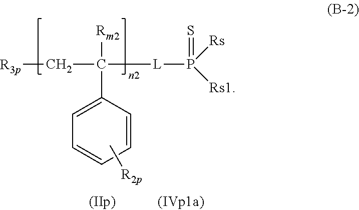

43.-50. (canceled)

51. The polymer of claim 1, which has structure (B-3),

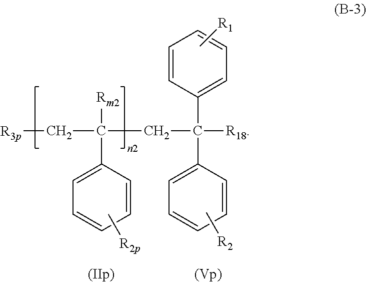

52. The polymer of claim 1, which has structure (B-3a)

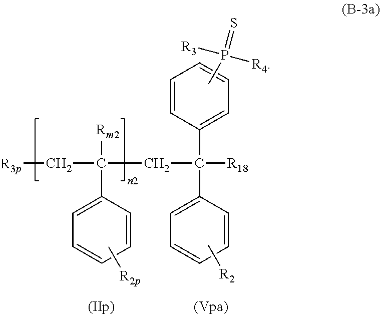

53.-54. (canceled)

55. The polymer of claim 1, which has structure (B-3d),

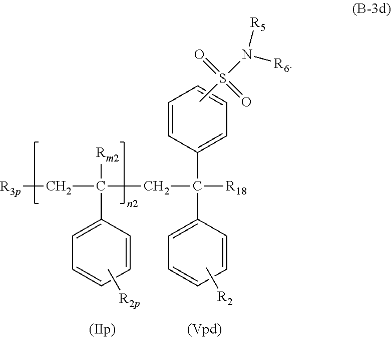

56.-57. (canceled)

58. The polymer of claim 1, which has structure (B-3g),

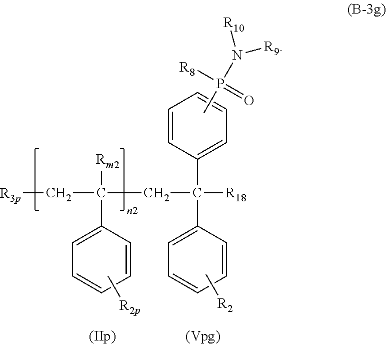

59.-60. (canceled)

61. A composition comprising a polymer of claim 1 and an organic spin casting solvent.

62. A composition comprising a polymer of claim 2 and an organic spin casting solvent.

63. A composition comprising a polymer of claim 3 and an organic spin casting solvent.

64. A composition comprising a polymer of claim 13 and an organic spin casting solvent.

65. A composition comprising a polymer of claim 22 and an organic spin casting solvent.

66. A composition comprising a polymer of claim 32, and an organic spin casting solvent.

67. A composition comprising a polymer of claim 33 and an organic spin casting solvent.

68. A composition comprising a polymer of claim 42 and an organic spin casting solvent.

69. A composition comprising a polymer of claim 51 and an organic spin casting solvent.

70. A process of forming a pinning layer brush selectively on a substrate which comprises both metallic surface areas and non-metallic surface areas, comprising the steps;
i) coating the composition of claim 61 on a said substrate forming a film,
ii) baking said film at a temperature from about 120° C. to about 250° C. for about 1 minute to about 1 hour to form a baked film,
iii) washing said baked film with a solvent to remove ungrafted polymer forming a pinning layer brush only on said metallic surface areas of said substrate.

71. (canceled)

72. A process comprising the steps;
ia) coating a composition according of claim 61 on a substrate which comprises both metallic surface areas and non-metallic surface areas forming a film,
iia) baking said film at a temperature from about 120° C. to about 250° C. for about 1 minute to about 1 hour to form a baked film,
iiia) washing said baked film with a solvent to remove ungrafted polymer forming a grafted substrate wherein pinning layer brush are only present on said metallic surface areas of said substrate,
iva) coating said grafted substrate with a neutral layer composition forming a neutral layer coating,
va) curing said neutral layer coating,
via) washing away, with a solvent, uncured neutral layer, leaving in said non-metallic areas a neutral directing brush, forming on said substrate a chemoepitaxy directing layer,
viia) coating on said chemoepitaxy directing layer with a block copolymer solution forming a coating of block copolymer,
viiia) annealing said coating of block copolymer to form a directed self-assembled film of the block copolymer on said chemoepitaxy directing layer.

73.-75. (canceled)

76. A compound of structure (1), wherein
R₁ is a chelating group located at the meta or para position selected from a phosphinothioic moiety of structure (Ia) an aminosulfonyl moiety of structure (Ib), a phosphonamide moiety of structure (Ic) wherein * designates the attachment point of these moieties to said compound of structure (1), wherein
in said phosphinothioic moiety of structure (Ia), R₃ and R₄ are independently an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy; and a dialkyl amino moiety, —N(R₉)(R₁₀), in which Re and R₁₀ are independently selected from a C-1 to C-8 linear alkyl, a C-3 to C-8 branched alkyl, a C-3 to C-8 cyclic alkyl,
in said aminosulfonyl moiety of structure (Ib), R₅ and R₆, are independently a C-1 to C-8 linear alkyl, a C-3 to C-8 branched alkyl, a C-3 to C-8 cyclic alkyl, and said dialkyl amino moiety, —N(R₉)(R₁₀),
in said phosphonamide moiety of structure (Ic), R₇ is said dialkyl amino moiety, —N(R₉)(R₁₀), and R₈ is selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy or a C-3 to C-8 cyclic alkyloxy,
R₂ is a substituent, located at the meta or para positions which is selected from the group consisting of H, an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; a phosphinothioic moiety of structure (Ia), an aminosulfonyl of structure (Ib), and a phosphonamide of structure (Ic)

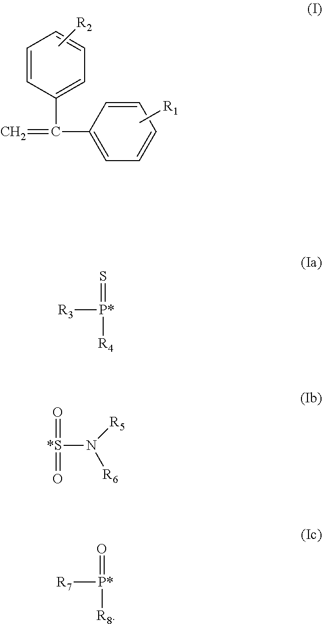

77.-95. (canceled)

96. A compound of structure (II), wherein
R₁₁ is a phosphinothioic moiety of structure (IIa), wherein * designates the attachment point of this moieties to said compound of structure (II), wherein
R₁₃ and R₁₄ are independently selected from the group consisting of an aryl, an alkylenearyl, a C-2 to C-8 alkyleneoxyalkyl, a C-2 to C-8 haloalkyl, a C-1 to C-8 linear alkyl, a C-3 to C-8 branched alkyl, a C-3 to C-8 cyclic alkyl, a C-1 to C-8 linear alkyloxy, a C-3 to C-8 branched alkyloxy, a C-3 to C-8 cyclic alkyloxy; and a dialkyl amino moiety, —N(R₉)(R₁₀), in which R₉ and R₁₀ are independently selected from a C-1 to C-8 linear alkyl, a C-3 to C-8 branched alkyl, a C-3 to C-8 cyclic alkyl;
L₁ is a linking moiety selected from the group consisting of a direct valence bond, a C-2 to C-8 alkylene moiety(-alkylene-), an arylene moiety (-aryl-), an alkyleneoxyaryl moiety (*-alkylene-O-aryl-**), an alkylenearyl moiety (*-alkylene-aryl-**), wherein * designates the attachment points of the L₁ organic linking moiety to the phosphorous in structure (IIa), and * designates where L₁ within the moiety R₁ is attached to the carbonyloxy of compound (II),
R₁₂ is H or a C-1 to C-4 alkyl,

97.-125. (canceled) [chemical structure]
